(19) United States
(12) Patent Application Publication
(10) Pub. No.: US 2023/0151098 A1
(43) Pub. Date: May 18, 2023

(54) Compositions and Methods for Vaccination and the Treatment of Infectious Diseases

(71) Applicant: Jounce Therapeutics, Inc., Cambridge, MA (US)

(72) Inventors: Christopher Harvey, Boston, MA (US); Richard Murray, Cambridge, MA (US); Robert Huebner, Cambridge, MA (US)

(73) Assignee: Jounce Therapeutics, Inc., Cambridge, MA (US)

(21) Appl. No.: 17/917,433

(22) PCT Filed: Apr. 19, 2021
(86) PCT No.: PCT/US2021/027908

§ 371 Date: Oct. 6, 2022

Related U.S. Application Data
(60) Provisional application No. 63/136,279, filed on Jan. 12, 2021.
(60) Provisional application No. 63/084,821, filed on Sep. 29, 2020.
(60) Provisional application No. 63/012,574, filed on Apr. 20, 2020.

(51) Int. Cl. C07K 16/28; A61P 31/00

(52) U.S. Cl. CPC C07K 16/2818; A61P 31/00; C07K 2317/75; A61K 2039/505

(57) ABSTRACT

Methods of treating infectious diseases, such as a viral diseases, and methods of enhancing the effectiveness of a vaccine against an infectious disease, such as a viral disease, with an ICOS agonist, such as an anti-ICOS agonist antibody, are provided.

### CROSS REFERENCE TO RELATED APPLICATIONS

This application claims the benefit of priority of U.S. Provisional Application Nos. 63/012,574, filed Apr. 20, 2020; 63/084,821, filed Sep. 29, 2020; and 63/136,279, filed Jan. 12, 2021; each of which is incorporated by reference in its entirety for any purpose.

### FIELD OF THE INVENTION

Methods of treating infectious diseases, such as a viral diseases, and methods of enhancing the effectiveness of a vaccine against an infectious disease, such as a viral disease, with an ICOS agonist, such as an anti-ICOS agonist antibody, are provided.

### BACKGROUND

ICOS is a member of the B7/CD28/CTLA-4 immunoglobulin superfamily and is specifically expressed on T cells. Unlike CD28, which is constitutively expressed on T cells and provides co-stimulatory signals necessary for full activation of resting T cells, ICOS is expressed only after initial T cell activation.

ICOS has been implicated in diverse aspects of T cell responses (reviewed in Simpson et al., 2010, Curr. Opin. Immunol., 22: 326-332). It plays a role in the formation of germinal centers, T/B cell collaboration, and immunoglobulin class switching. ICOS-deficient mice show impaired germinal center formation and have decreased production of interleukin IL-10.

These defects have been specifically linked to deficiencies in T follicular helper cells.

ICOS also plays a role in the development and function of other T cell subsets, including Th1, Th2, and Th17. Notably, ICOS co-stimulates T cell proliferation and cytokine secretion associated with both Th1 and Th2 cells. Accordingly, ICOS KO mice demonstrate impaired development of autoimmune phenotypes in a variety of disease models, including diabetes (Th1), airway inflammation (Th2) and EAE neuro-inflammatory models (Th17).

In addition to its role in modulating T effector (Teff) cell function, ICOS also modulates T regulatory cells (Tregs). ICOS is expressed at high levels on Tregs, and has been implicated in Treg homeostasis and function.

Upon activation, ICOS, a disulfide-linked homodimer, induces a signal through the PI3K and AKT pathways. Subsequent signaling events result in expression of lineage specific transcription factors (e.g., T-bet, GATA-3) and, in turn, effects on T cell proliferation and survival.

ICOS ligand (ICOSL; B7-H2; B7RP1; CD275; GL50), also a member of the B7 superfamily, is the only ligand for ICOS and is expressed on the cell surface of B cells, macrophages and dendritic cells. ICOSL functions as a non-covalently linked homodimer on the cell surface in its interaction with ICOS. Human ICOSL, although not mouse ICOSL, has been reported to bind to human CD28 and CTLA-4 (Yao et al., 2011, Immunity, 34: 729-740).

### SUMMARY

In some embodiments, methods of treating an infectious disease in a subject are provided, said method comprising administering a therapeutically effective amount of an ICOS agonist to said subject.

In some embodiments, methods of enhancing the effectiveness of a vaccine against an infectious disease in a subject are provided, said method comprising administering a therapeutically effective amount of an ICOS agonist to said subject concurrently with or after administration of the vaccine to the subject. In some embodiments, the ICOS agonist is administered after administration of the vaccine to the subject, such as after administration of a complete dose of vaccine. In some embodiments, the ICOS agonist is administered concurrently with administration of the vaccine to the subject, such as concurrently with administration of each dose of vaccine.

In some embodiments, the infectious disease is a bacterial disease. In some such embodiments, the bacterial disease is caused by infection with a bacteria selected from Staphylococcus aureus, Pseudomonas aeruginosa, Streptococcus pneumoniae, Corynebacterium diphtheriae (diphtheria), Clostridium tetani (tetanus), Borrelia bacterium (Lyme disease), Clostridium difficile, and Bordetella pertussis. In some embodiments, the bacterial disease is caused by infection with Streptococcuspneumoniae. In some embodiments, the vaccine is PneumoVax 34 (Merck). In some embodiments, the subject is at least 65 years old; or wherein the subject is younger than 65 years old and has a chronic illness, diabetes, alcoholism, weakened immune system, cochlear implants, CSF leaks, and/or is a smoker. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In some embodiments, said infectious disease is a viral disease caused by an influenza virus (e.g., influenza virus A, B, C, or D). In some such embodiments, the vaccine is a seasonal influenza vaccine or a pandemic influenza vaccine. In some embodiments, the subject is at least 50 years old, at least 60 years old, or at least 65 years old. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In some embodiments, said infectious disease is a viral disease caused by varicella zoster virus. In some embodiments, the vaccine is Zostavax (Merck). In some embodiments, the subject is at least 50 years old, at least 60 years old, or at least 65 years old. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In various embodiments, said infectious disease is a viral disease. In some embodiments, said viral disease is caused by infection with a virus selected from an human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus. In some embodiments, said virus is a coronavirus selected from MERS-CoV, SARS-CoV, and SARS-CoV-2. In some embodiments, said virus is SARS-CoV-2. In some embodiments, said subject has tested positive for SARS-CoV-2. In some embodiments, said subject has not exhibited symptoms of SARS-CoV-2 infection prior to said administering of the ICOS agonist. In some embodiments, said subject has exhibited one or more symptoms of SARS-CoV-2, such as one or more of fever, cough, shortness of breath, loss of sense of taste and/or smell, fatigue, and body aches, prior to administering of the ICOS agonist. In some embodiments, said symptoms include coughing, fever, and/or shortness of breath. In some embodiments, the subject is at least 50 years old, at least 60 years old, or at least 65 years old. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In various embodiments, wherein said ICOS agonist is an anti-ICOS agonist antibody. In some such embodiments, said anti-ICOS agonist antibody is selected from vopratelimab, BMS-986226, KY1044, KY1055, and GSK3359609, or an antibody comprising the heavy and light chain CDRs or the heavy and light chain variable regions of vopratelimab, BMS-986226, KY1044, KY1055, or GSK3359609.

In some embodiments, the ICOS agonist antibody comprises an HCDR1 comprising the amino acid sequence of SEQ ID NO: 9; an HCDR2 comprising the amino acid sequence of SEQ ID NO: 10; an HCDR3 comprising the amino acid sequence of SEQ ID NO: 11; an LCDR1 comprising the amino acid sequence of SEQ ID NO: 12; an LCDR2 comprising the amino acid sequence of SEQ ID NO: 13; and an LCDR3 comprising the amino acid sequence of SEQ ID NO: 14.

In some embodiments, the ICOS agonist antibody comprises a heavy chain variable region (VH) and a light chain variable region (VL), wherein the VH is at least 90%, 91%, 92%, 93%, 94%, 95%, 96%, 97%, 98%, 99%, or 100% identical to the amino acid sequence of SEQ ID NO: 7 and the VL is at least 90%, 91%, 92%, 93%, 94%, 95%, 96%, 97%, 98%, 99%, or 100% identical to the amino acid sequence of SEQ ID NO: 8.

In some embodiments, the ICOS agonist antibody comprises a VH comprising the amino acid sequence of SEQ ID NO: 7 and a VL comprises the amino acid sequence of SEQ ID NO: 8. In some embodiments, the antibody comprises a heavy chain comprising the amino acid sequence of SEQ ID NO: 15 and a light chain comprising the amino acid sequence of SEQ ID NO: 16. In some embodiments, the ICOS agonist antibody comprises a heavy chain comprising the amino acid sequence of SEQ ID NO: 17 and a light chain comprising the amino acid sequence of SEQ ID NO: 16.

### BRIEF DESCRIPTION OF THE DRAWINGS

FIGS. 1A and 1B show the profiling of ICOS-high CD4 T cells (of ICOS^{hi}CD4⁺ cells) from cancer patients. FIG. 1A, left panel, shows that ICOS^{hi}CD4⁺ cells express the Th1 transcription factor Tbet. FIG. 1A, right panel, shows that ICOS^{hi}CD4⁺ cells do not express CD25 and FoxP3. FIG. 1B shows the change in mRNA levels for T cell markers measured in the ICOS^{hi}CD4⁺ population and ICOS low CD4 T cell (ICOS^{lo}CD4⁺) population. In general, a log 2 fold increase of 0 to 1.5 was observed in the T cell marker mRNAs in ICOS^{hi}CD4⁺ subjects while a log 2 fold decrease of 0 to 1.5 was observed in the ICOS^{lo}CD4⁺ subjects.

FIG. 2 shows that vopratelimab stimulates ICOS^{hi}CD4⁺ cells after sub-optimal T cell receptor (TCR) signal. The left panel shows that ICOS^{lo}CD4⁺ cells did not respond to vopratelimab treatment. The middle panel shows the mixed population of ICOS^{hi}CD4⁺ and ICOS^{lo}CD4⁺ cells. The right panel shows that ICOS^{hi}CD4⁺ proliferate in a dose dependent manner when treated with vopratelimab.

FIG. 3 shows that vopratelimab stimulates antigen-specific ICOS^{hi}CD4⁺ cells in an ex vivo tetanus toxoid recall assay. Following vopratelimab treatment, ICOS^{hi}CD4⁺ cells showed significant increased IFNγ and TNFα expression (right panel) while ICOS^{lo}CD4⁺ did not (left panel). The middle panel shows the mixed population of ICOS^{hi}CD4⁺ and ICOS^{lo}CD4⁺ cells.

FIG. 4 shows that vopratelimab stimulates antigen-specific ICOS^{hi}CD4⁺ cells in an ex vivo CMV recall assay. Following vopratelimab treatment, ICOS^{hi}CD4⁺ cells showed significant increased IFNγ and TNFα expression (right panel) while ICOS^{lo}CD4⁺ did not (left panel). The middle panel shows the mixed population of ICOS^{hi}CD4⁺ and ICOS^{lo}CD4⁺ cells.

FIG. 5 shows a schematic of how CD4 T cells support vaccine responses via humoral immunity. ICOS is essential in the differentiation and maintenance of CD4 Tfh cells. Circulating Tfh (cTfh) cells can be detected in the blood and maintain an ICOS^{hi} phenotype. Activated cTfh cells (i.e., ICOS^{hi}PD-1⁺) are induced by vaccination and correlate with increased antibody responses and expansion of pathogen-specific memory B cells.

FIGS. 6A and 6B show that ICOS agonist administration increases frequency of Tfh and B cells and enhances antibody responses in an NP-OVA vaccine model. As shown in FIG. 6A, mice were treated with NP-OVA and isotype matched control antibody, ICOS agonist antibody, or ICOS ligand (ICOS-L) at 24 hours prior to and at 48 hours after vaccination. Mice were sacrificed 5 days following administration of the last treatment dose, with blood and draining lymph nodes (dLN) harvested. Tfh and B cell responses were assessed by flow cytometry, and OVA-specific antibodies were quantified by ELISA. As shown in FIG. 6B, treatment with ICOS agonist antibody resulted in an increase in Tfh and B cell numbers within draining lymph nodes (dNL), as measured by the percentage of CD19+ in CD15+ cells in dLN for B cells and percentage of PD-1+ Bc16+ in CD4+ Foxp3− cells for Tfh cells, (top panels) as well as elevated serum IgG2a and IgG2b levels relative to both naïve and isotype treated animals (bottom panels). “Vax” stands for NP-OVA vaccine.

FIG. 7 shows a schematic the mechanism of action of vopratelimab and ICOS^{hi}CD4⁺ cells.

FIGS. 8A and 8B are graphs showing antibody binding to the indicated proteins. Mice were immunized with linear sequence of SARS-CoV-2 spike protein and treated as indicated. Antibodies specific to conformational spike protein (FIG. 8A) and linear spike protein (FIG. 8B) were detected by ELISA.

FIG. 9 is a violin plot showing antibody binding to linear of SARS-CoV-2 spike protein. Mice were immunized against linear SARS-CoV-2 spike protein and treated as indicated. Antibodies specific to linear spike protein (FIG. 8B) were detected by ELISA.

### DETAILED DESCRIPTION OF SOME EMBODIMENTS

In general, the present application provides a method of treating infectious diseases such as viral diseases and enhancing the effectiveness of a vaccine against infectious diseases such as viral diseases by administering an ICOS agonist. In some embodiments the ICOS agonist is an antibody. Exemplary ICOS agonist antibodies include vopratelimab, BMS-986226, and GSK3359609, or antibodies comprising the CDRs or variable domain sequences of one of those antibodies. In some embodiments, the ICOS agonist is administered to enhance the effectiveness of a vaccine against infectious diseases in a subject with a partially or wholly compromised immune system, e.g., due to the effects of aging or an immune disease. In some embodiments, the subject is at least 50 years old, at least 60 years old, or at least 65 years old. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia. In such embodiments, the invention is based, in part, on the hypothesis that the an ICOS agonist could sensitize the subject to vaccination by compensating for lower amounts of ICOS ligand or to induce expansion of desired immune cells, whereby the ICOS agonist enhances the effectiveness of the vaccine and partially or completely restores the sensitivity of said subjects to the vaccine.

In some embodiments, the antibody is vopratelimab. Vopratelimab may specifically bind to ICOS and stimulate antigen experienced CD4 cells through the AKT pathway. In clinical studies in cancer patients, vopratelimab has been shown to stimulate and maintain an activated ICOS^{hi}CD4⁺ population throughout deep durable tumor response lasting over 2 years. (See Carthon et al. Clin. Can. Res. (2010); Harvey, C., AACR (2019); Hanson, A., SITC (2018).) Patients that have detectable levels of this cell population in the blood, in some cases, may experience markedly improved clinical benefit. Analysis of this cell population has established further characteristics of these cells as an oligoclonal population of Th1, central memory, and T follicular helper (Tfh) cells that do not display markers for T regulatory cells. Vopratelimab has been shown to stimulate the antigen experienced CD4 cells when they are in an ICOS^{hi} state, consistent with a biological role as an immune co-stimulator. CD4⁺ Tfh may be critical, both in germinal centers (GC Tfh) and circulating counterparts (cTfh), for productive humoral responses. Human Tfh cells show elevated expression levels of ICOS.

Studies on influenza vaccination have also shown an association of the emergence of the cTfh cells in the blood post vaccination and a productive humoral response to the virus. (See Ciabattini et al. Frontiers in Immunology (2013); Koutsakos et al. J. Immunol (2019); McAdam et al. Nature (2001).) In mice, these similar cells have been shown to block influenza infection in pre-clinical models.

In a SARS-CoV-1 infection model in mice, CD4⁺ T cells were shown to be required to limit the associated lung pathology and to neutralize antibody production against the virus. (J. Chen et al. J. Virology 84(3): 1289-1301 (2010).) A recent publication describing an immune response in a COVID-19 patient, who was ill and then recovered, showed the emergence of ICOS+(or ICOS^{hi}) Tfh in the blood prior to and throughout recovery. (I. Thevarajan et al. Nature Medicine published online Mar. 16, 2020, available at https doi.org 10.1038/s41591-020-0819-2.) Furthermore, humans deficient for the ICOS receptor have been shown to have defective humoral immunity and germinal centers.

An ICOS⁺ (or ICOS^{hi}) CD4⁺ Tfh-like population of cells, which emerges based on antigen stimulation of CD4⁺ cells, represents a potentially universal mechanism of a productive immune response. Selectively stimulating these cells when they are in an ICOS+/ICOS^{hi} state with an ICOS agonist may allow for the acceleration of, or for a more extensive, humoral response to infectious agent antigens. Accordingly, without being bound by this mechanistic hypothesis, selective stimulation of ICOS+/ICOS^{hi} CD4 T cells may represent a universal immune amplifier, relevant to infectious disease scenarios where a productive immune response depends upon, or is influenced by, CD4⁺ cells with high levels of ICOS.

The section headings used herein are for organizational purposes only and are not to be construed as limiting the subject matter described. All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993); and updated versions thereof.

### L Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se.

For example, description referring to “about X” includes description of “X”.

“ICOS” and “inducible T-cell costimulatory” as used herein refer to any native ICOS that results from expression and processing of ICOS in a cell. The term includes ICOS from any vertebrate source, including mammals such as primates (e.g., humans and cynomolgus monkeys) and rodents (e.g., mice and rats), unless otherwise indicated. The term also includes naturally occurring variants of ICOS, e.g., splice variants or allelic variants. The amino acid sequence of an exemplary human ICOS precursor protein, with signal sequence (with signal sequence, amino acids 1-20) is shown in SEQ ID NO: 1. The amino acid sequence of an exemplary mature human ICOS is shown in SEQ ID NO: 2. The amino acid sequence of an exemplary mouse ICOS precursor protein, with signal sequence (with signal sequence, amino acids 1-20) is shown in SEQ ID NO: 3. The amino acid sequence of an exemplary mature mouse ICOS is shown in SEQ ID NO: 4. The amino acid sequence of an exemplary cynomolgus monkey ICOS precursor protein, with signal sequence (with signal sequence, amino acids 1-20) is shown in SEQ ID NO: 5. The amino acid sequence of an exemplary mature cynomolgus monkey ICOS is shown in SEQ ID NO: 6.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an ICOS epitope is an antibody that binds this epitope with greater affinity, avidity, more readily, and/or with greater duration than it binds to other ICOS epitopes or non-ICOS epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” herein is used in the broadest sense and encompasses various antibody structures, including but not limited to monoclonal antibodies, polyclonal antibodies, multispecific antibodies (for example, bispecific(such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a 74 constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, CL. Nonlimiting exemplary light chain constant regions include X and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

In some embodiments, the “K_{D},” “K_{d},” “Kd” or “Kd value” of the antibody is measured by using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, for example, Chen et al., J Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51:19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is ForteBio Octet® RED96 system (Pall Corporation). See, e.g., Abdiche et al., 2008, Anal. Biochem. 377: 209-277.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity. In some embodiments, biological activity of an ICOS protein includes, for example, costimulation of T cell proliferation and cytokine secretion associated with Th1 and Th2 cells; modulation of Treg cells; effects on T cell differentiation including modulation of transcription factor gene expression; induction of signaling through PI3K and AKT pathways; and mediating ADCC.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; C1q binding; CDC; ADCC; phagocytosis; down regulation of cell surface receptors (for example B cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (for example B cell receptor); and B cell activation.

“Human effector cells” are leukocytes which express one or more FcRs and perform effector functions. In some embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, for example, from blood.

“Antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362; 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538; 7,994,290.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, for example, as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, for example, in U.S. Pat. No. 6,194,551 B1, U.S. Pat. Nos. 7,923,538, 7,994,290 and WO 1999/51642. See also, for example, Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In some embodiments, a reference sample, reference cell or reference tissue is obtained from a healthy part of the body of the same subject or patient in whom a disease or condition is being identified using a composition or method of the invention. In some embodiments, a reference sample, reference cell or reference tissue is obtained from a healthy part of the body of one or more individuals who are not the subject or patient in whom a disease or condition is being identified using a composition or method of the invention.

A “disease” or “disorder,” as used herein, refers to a condition where treatment is needed and/or desired.

An “infectious disease,” as used herein, refers to a disease or disorder that is caused by presence of a virus or by a pathogenic microorganism, such as bacteria, parasites or fungi in the body. An infectious disease can spread, directly or indirectly, from one infected subject (“host”) to another. In some cases, the subject may experience one or more phenotypic effects from the presence of the virus or viral particles or microorganism, such as fever, aches, pains, loss of smell or taste, fatigue, sinus congestion, digestive upset, and/or respiratory symptoms (e.g. coughing, shortness of breath, sore throat, etc.). In other instances, the disease may be asymptomatic.

A “viral infection” or “viral disease” as used herein is an infectious disease caused by the presence of virus or viral particles in the body. In some embodiments, the viral disease is caused by an human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus. In some embodiments, the viral disease is caused by a coronavirus. In some embodiments, the coronavirus is MERS-CoV, SARS-CoV or SARS-CoV-2. In some embodiments, the viral disease is caused by an influenza virus. In some embodiments, the influenza virus is influenza virus A, influenza virus B, influenza virus C or influenza virus D.

A “vaccine,” as used herein, refers to a therapeutic preparation comprising proteins and/or nucleic acid molecules from a virus or pathogenic microorganisms, killed microorganisms, living attenuated organisms, or living fully virulent viruses or microorganisms that is used to stimulate the production of antibodies or to increase immunity to an infectious disease. In some embodiments, a vaccine is used to prevent onset of disease, for example in a subject who is susceptible to developing the disease. In some embodiments, a vaccine is used to treat a disease after a subject has the illness.

As used herein, when more than one drug or therapy is administered to a patient, the two drugs or therapies may be administered “concurrently” or “sequentially.” As used herein, “concurrently” means that the drugs or therapies are administered together, such as at the same time, or in the same medical visit. As used herein, “sequentially” means that the drugs or therapies are administered one after the other, such as a few minutes, a few hours, or a few days apart, or on different medical visits.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. In the case of an infectious disease, the aim of treatment may be to minimize symptoms and infectivity, and/or shorten the duration of the infection, or to prevent onset of the disease. The methods provided herein contemplate any one or more of these aspects of treatment. In-line with the above, the term treatment does not require one-hundred percent removal of all aspects of the disease. “Treating” in the case of a viral disease includes any or all of preventing or minimizing symptoms and infectivity, and/or shortening the duration of the infection, which may include inhibiting viral attachment, penetration, uncoating, replication, assembly and release.

“Ameliorating” means a lessening or improvement of one or more symptoms as compared to not administering an ICOS agonist antibody. “Ameliorating” also includes shortening or reduction in duration of a symptom.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “target engagement” or “TE,” means herein a measure for the degree of occupation of ICOS by an ICOS agonist antibody. The target engagement level can be measured by availability of free-receptor using ICOS agonist antibodies, and can be determined by methods known in the art, and in some embodiments, is determined by a method disclosed herein. The target engagement level can be determined as percentage (%) relative to a control, which may be, e.g., a peripheral blood sample from the subject being treated, but before treatment with the ICOS agonist antibody. Alternatively, the control can be a level from a matched sample (e.g., a peripheral blood sample) of a healthy individual. In some embodiments, the target engagement level of an ICOS agonist antibody is measured in a peripheral blood sample (e.g., PBMCs). In some such embodiments, upon treatment with an ICOS agonist antibody, the number of ICOS receptors on the surface of T lymphocytes that are free (i.e., not bound to antibody) may be quantified. Decrease in observed available receptors may serve as an indication that ICOS agonist antibodies are binding to ICOS.

An “expected target engagement level” means herein an experimentally determined average level of target engagement exhibited in subjects after a defined amount of time following administration of a particular dose of ICOS agonist antibody. To calculate an “expected target engagement level” for a particular dosage and time, the target engagement level as measured in at least three reference subjects at the particular time after administration of the ICOS agonist antibody at the particular dose, is summed and divided by the number of reference subjects. In some embodiments, the expected target engagement level is measured in at least five reference subjects. In some embodiments, target engagement level is determined in a sample of PBMCs from one or more subjects. Target engagement may be determined by methods known in the art, and in some embodiments, is determined by a method disclosed herein. In some embodiments, a subsequent dose of ICOS agonist antibody is administered once the expected target engagement level of the ICOS agonist antibody in peripheral blood of the subject is less than about 70%, less than about 60%, less than about 50%, less than about 40%, less than about 30%, or less than about 20%. In some embodiments, the subsequent dose is administered when the expected target engagement level of the ICOS agonist antibody is greater than about 10%, or greater than about 15%, or greater than about 20%. In certain embodiements, the subsequent dose is administered once the expected target engagement level of the ICOS agonist antibody in peripheral blood of the subject is equal to or less than about 20%, equal to or less than about 10%, equal to or less than about 5%, or is 0%.

In some embodiments, the terms “elevated levels of ICOS,” “elevated ICOS levels,” “ICOS at an elevated level,” “ICOS^{HIGH},” and “ICOS^{hi}” refer to increased levels of ICOS in cells (e.g., CD4⁺ T cells) of a subject, e.g., in a peripheral blood sample of the subject. The elevated levels can be determined relative to a control, which may be, e.g., a peripheral blood sample from the same subject at an earlier timepoint. Alternatively, the control can be a level from a matched sample (e.g., a peripheral blood sample) of a healthy individual. In some embodiments, the level of ICOS is determined at the level of expressed protein, which may be detected in some embodiments using an antibody directed to an intracellular portion of ICOS. In some embodiments, the detection using such an antibody is done by use of flow cytometry. In some embodiments, an increase of at least 2-fold (e.g., at least 3-fold, 4-fold, 5-fold, 7.5-fold, 10-fold, or 15-fold) in mean fluorescence intensity (MFI), relative to a control, indicates detection of elevated ICOS levels. In some embodiments, detection of an increase in ICOS levels in at least 5% (e.g., at least 10%, 20%, 30%, 40%, 50%, 60%, 70%, 80%, or 90%) of CD4⁺ T cells in a peripheral blood sample indicates a subject having an ICOS^{hi} sample. In some embodiments, an increase of at least 2-fold (e.g., at least 3-fold, 4-fold, 5-fold, 7.5-fold, 10-fold, or 15-fold) in mean fluorescence intensity (MFI), relative to a control, in at least 5% (e.g., at least 10%, 20%, 30%, 40%, 50%, 60%, 70%, 80%, or 90%) of CD4⁺ T cells in a peripheral blood sample indicates detection of elevated ICOS levels. In some embodiments, elevated ICOS levels refer to an increase in total ICOS expression levels (e.g., mRNA levels or protein levels) in CD4⁺ T cells in the peripheral blood test sample of about 10%, 20%, 30%, 40%, 50%, 60%, 70%, 80%, 85%, 90%, 95%, 100%, or greater relative to a control sample. In some embodiments, elevated ICOS levels refers to an increase in total ICOS expression levels (e.g., mRNA levels or protein levels) in the CD4⁺ T cells in a peripheral blood sample of about at least 1.1×, 2×, 3×, 4×, 5×, 10×, 15×, 20×, 30×, 40×, 50×, 100×, 500×, 1000×, or greater relative to a control sample.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. Unless otherwise specified, the terms “reduce”, “inhibit”, or “prevent” do not denote or require complete prevention over all time.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased on non-treated sample of a subject individual. In some examples, a reference is obtained from one or more healthy individuals who are not the subject or patient.

As used herein, “delaying development of a disease” means to defer, hinder, slow, retard, stabilize, suppress and/or postpone development of the disease (such as a viral infection). This delay can be of varying lengths of time, depending on the history of the disease and/or individual being treated. As is evident to one skilled in the art, a sufficient or significant delay can, in effect, encompass prevention, in that the individual does not develop the disease.

As used herein, to “suppress” a function or activity is to reduce the function or activity when compared to otherwise same conditions except for a condition or parameter of interest, or alternatively, as compared to another condition.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at doses and for periods of time necessary, to achieve the desired therapeutic and/or prophylactic result.

A “prophylactically effective amount” refers to an amount effective, at doses and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the doses and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dose, administration, combination therapy, contraindications and/or warnings concerning the use of such therapeutic products. These are also referred to as the Full Prescribing Information for a product in the U.S.

An “article of manufacture” is any manufacture (for example, a package or container) or kit comprising at least one reagent, for example, a medicament for treatment of a disease or disorder (for example, a viral infection), or a probe for specifically detecting a biomarker described herein. In some embodiments, the manufacture or kit is promoted, distributed, or sold as a unit for performing the methods described herein.

The term “prediction” is used herein to refer to the likelihood that a subject will respond either favorably or unfavorably to a therapeutic agent or combination of therapeutic agents. In some embodiments, the prediction relates to the extent of those responses. In some embodiments, the methods of prediction described herein can be used to make treatment decisions by choosing the most appropriate treatment modalities for a particular subject.

### II. Therapeutic Methods

This disclosure relates to methods of treating disease in a subject in need of such treatment comprising administering an ICOS agonist antibody to the subject. The disclosure also relates to methods of enhancing the effectiveness of a vaccine against an infectious disease in a subject comprising administering an ICOS agonist concurrently with or after administration of the vaccine. In some embodiments, the ICOS agonist is an antibody. In some embodiments, the ICOS agonist antibody is vopratelimab, BMS-986226, KY1044, KY1055, or GSK3359609, or an antibody comprising the CDR or variable region (VH and VL) sequences of one of those antibodies.

Patients that can be treated as described herein comprise patients with an infectious disease. In some cases, the disease is a viral disease. Exemplary types of viral disease include, but are not limited to viral disease caused by human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus. In some embodiments, the viral disease is caused by a coronavirus. In some embodiments, the coronavirus is MERS-CoV, SARS-CoV or SARS-CoV-2. In some embodiments, the disease is COVID-19 (i.e., viral disease caused by SARS-CoV-2). In some embodiments, the viral disease is caused by an influenza virus. In some embodiments, the influenza virus is influenza virus A, influenza virus B, influenza virus C or influenza virus D. In other embodiments, the infectious disease is caused by a pathogenic microorganism, such as bacteria, protozoa, fungi, etc. Nonlimiting exemplary bacterial diseases include diseases caused by Staphylococcus aureus, Pseudomonas aeruginosa, Streptococcus pneumoniae, Corynebacterium diphtheriae (diphtheria), Clostridium tetani (tetanus), Borrelia bacterium (Lyme disease), Clostridium difficile, and Bordetella pertussis.

A study by Thevarajan et al. Nature Medicine (2020) highlighted the developing immune response of a patient who was infected with SARS-CoV-2 and recovered from symptoms of COVID-19, the disease caused by the SARS-CoV-2 virus. ICOS+(or ICOS^{hi}) Tfh emerged in the patient's blood prior to and throughout recovery. Previously, in a SARS-CoV-1 infection model in mice, CD4⁺ T cells were shown to be required to limit the associated lung pathology and for neutralizing antibody production to the virus. ICOS^{hi} cells, which emerge based on antigen stimulation of CD4⁺ cells, represent a potentially universal mechanism of a productive immune response. (Chen et al., J. Virology (2010).) Selectively stimulating ICOS' cells with an ICOS agonist might allow for the acceleration of, or for a more extensive, humoral response to infectious agent antigens. Thus, stimulating ICOS^{hi} cells with an ICOS agonist may help to increase an immune response in disease scenarios where a productive immune response depends upon, or is influenced by, CD4⁺ cells with high levels of ICOS. Similarly, stimulating ICOS^{hi} cells with an ICOS agonist may help to improve immune responses to vaccines against such infectious diseases.

In some embodiments, the viral disease is caused by SARS-CoV-2. In some embodiments, the subject has tested positive for SARS-CoV2. In some embodiments, the subject has exhibited symptoms of SARS-CoV-2 infection (also called COVID-19). In some embodiments, the subject has not exhibited symptoms of SARS-CoV-2 infection prior to administration of ICOS agonist antibody. Exemplary symptoms include, for example, fever, coughing, shortness of breath, fatigue, body aches, digestive upset, and/or loss of sense of smell and/or taste. In some embodiments, the subject has symptoms that comprise coughing, fever and/or shortness of breath. In some embodiments, the subject to be treated is at least 60 years old, at least 65 years old, at least 70 years old, at least 75 years old, or at least 80 years old.

Patients that can be treated as described herein include patients who have not previously received an infectious disease therapy and patients who have received previous (e.g., 1, 2, 3, 4, 5, or more) doses or cycles of one or more (e.g., 1, 2, 3, 4, 5, or more) infectious disease therapies, as well as patients who have received a vaccine therapy. In some embodiments, the treated patients have not responded to or have not shown adequate response to a previously administered therapy. In some embodiments, the treated patients have or have been diagnosed with an immune disorder selected from Wiskott-Aldrich syndrome, X-linked thrombocytopenia (XLT), CVID, hypogammaglobulinemia, and/or idiopathic CD4 lymphopenia.

ICOS agonists such as ICOS agonist antibodies can be administered alone or with other modes of infectious disease therapies. In some embodiments, an ICOS agonist antibody is administered in conjunction with another anti-viral agent. In some embodiments, the ICOS agonist antibody is administered with a second therapeutic method for treatment. In some embodiments, the ICOS agonist is administered in conjunction with or after treatment with a vaccine therapy. Thus, the administration of an ICOS agonist provided herein can be in combination with another system of treatment.

In some embodiments, a method of treating an infectious disease, such as a viral disease, is provided, wherein cells within a sample of the infected cells express ICOS. In some such embodiments, the infected cells may be considered to be ICOS-positive, ICOS+, ICOS^{Hi}, or to express ICOS. Expression of ICOS may be determined by, for example, by IHC.

Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of an ICOS agonist antibody is administered to a subject one or more times. In some embodiments, the ICOS agonist antibody is administered only once. In some embodiments, the effective dose of an ICOS agonist antibody may be administered multiple times, including for periods of at least a week, at least two weeks, at least a month, at least two months, at least three months, at least six months, at least a year, or at least two years. In some embodiments, the ICOS agonist antibody is administered once every three weeks. In other embodiments, the ICOS agonist antibody is administered once every two weeks, or once each week. In some embodiments, the timing of ICOS agonist antibody administration is determined based on the expected target engagement level, as described below, and the expected course of the disease.

In some embodiments, pharmaceutical compositions comprising the ICOS agonist antibody are administered in an amount effective for treatment of (including prophylaxis of) infectious disease, such as a viral disease. The therapeutically effective amount is typically dependent on the weight of the subject being treated, his or her physical or health condition, the extensiveness of the condition to be treated, or the age of the subject being treated.

The therapeutically effective amount is, in some embodiments, 0.01 mg/kg, 0.03 mg/kg, 0.1 mg/kg or 0.3 mg/kg. In some embodiments, the therapeutically effective amount is from 0.03 mg/kg to 0.3 mg/kg. In some embodiments, the therapeutically effective amount is from 0.03 mg/kg to 0.1 mg/kg. In some embodiments, the therapeutically effective amount is between 0.1 mg/kg and 0.3 mg/kg. In some embodiments, the therapeutically effective amount is 0.1 mg/kg. In some embodiments, the therapeutically effective amount is 0.03 mg/kg. In some embodiments, the therapeutically effective amount is 0.03, 0.04, 0.05, 0.06, 0.07. 0.08, 0.09, or 0.1 mg/kg.

Pharmaceutical compositions may also be administered in an amount effective for increasing the number of Teff cells; activating Teff cells; depleting Treg cells; and/or increasing the ratio of Teff cells to Treg cells. In some embodiments, the Treg cells are CD4⁺ FoxP3+ T cells. In some embodiments, the Teff cells are CD8+ T cells. In some embodiments, the Teff cells are CD4+ FoxP3− T cells and CD8+ T cells. In some embodiments, the Teff cells are CD4+ FoxP3− T cells.

In some embodiments, treatment with ICOS agonist antibody results in a pharmacodynamics readout, such as up-regulation of ICOS ligand (ICOSL). In some embodiments, up-regulation of ICOSL is observed on the surface of B cells. In some embodiments, up-regulation of ICOSL is observed on the surface of granulocytes. In some embodiments, up-regulation of ICOSL is observed on the surface of neutrophils. Up-regulation of ICOSL may be observed on cells in the spleen or on cells in peripheral blood. Up-regulation of ICOSL on the cell surface can be detected, for example, by flow cytometry. In some embodiments, soluble ICOSL is increased in the serum following treatment with ICOS agonist antibody. Soluble ICOSL can be detected by methods including, but not limited to, ELISA, MSD, and mass spectrometry.

In some embodiments, ICOS target engagement, as measured by availability of free-receptor to ICOS agonist antibodies, may be used as a pharmacodynamics readout. In some such embodiments, upon treatment by an ICOS agonist antibody, the number of ICOS receptors on the surface of T cells that are free to bind additional antibodies may be quantified. Decrease in observed available receptors may serve as an indication that ICOS agonist antibodies are binding ICOS on the surface of the cells.

In some embodiments, target engagement is measured on live CD4⁺ T cells in a sample taken from a subject that has been administered an ICOS agonist antibody. In some such embodiments, a labeled version of the treatment antibody (such as, for example, vopratelimab-DyLight 650) is used to detect free ICOS. Receptor availability may be determined using the following formula (where V stands for vopratelimab):

$$\% Receptor Available at time t = \frac{\begin{matrix}( MFI of VmG 2 a - mG 2 aDy 650 at time t - \\ MFI of isotypeDy 650 at time t )\end{matrix}}{\begin{matrix}( MFI of VmG 2 a - mG 2 aDy 650 prestudy - \\ MFI of isotypeDy 650 prestudy )\end{matrix}} \times 100$$

Target engagement is determined as 100%−% Receptor availability.

In some embodiments, a dose of an ICOS agonist, such as an ICOS agonist antibody, is administered when the expected target engagement level of the ICOS agonist in the peripheral blood of the subject is less than about 70%, or less than about 60%, or less than about 50%, or less than about 40%, or less than about 30%, or less than about 20%. In some embodiments, a subsequent dose of an ICOS agonist antibody is administered when the expected target engagement level of the ICOS agonist antibody in the peripheral blood of the subject is greater than about 10%, greater than about 15%, or greater than about 20%. In certain embodiments, a subsequent dose of an ICOS agonist antibody is administered when the expected target engagement level of the ICOS agonist antibody in the peripheral blood of the subject is equal to or less than about 20%, equal to or less than about 10%, equal to or less than about 5%, or is 0%. In certain embodiments, each dose of the ICOS agonist antibody is administered in an amount such that expected target engagement level of the ICOS agonist antibody in the peripheral blood of the subject immediately prior to a subsequent administration of said ICOS agonist antibody is from about 0% to about 50%, from about 0% to about 40%, from about 0% to about 30%, from about 0% to about 20%, from about 0% to about 10%, from about 0% to about 5%, or from about 5% to about 50%, from about 5% to about 40%, from about 5% to about 30%, from about 5% to about 20%, from about 5% to about 10%, from about 10% to about 70%, from about 10% to about 60%, from about 10% to about 50%, from about 10% to about 40%, from about 10% to about 30%, from about 10% to about 20%, from about 15% to about 70%, from about 15% to about 60%, from about 15% to about 50%, from about 15% to about 40%, from about 15% to about 30%, from about 15% to about 20%, from about 20% to about 70%, from about 20% to about 60%, from about 20% to about 50%, from about 20% to about 40%, or from about 20% to about 30%.

In some embodiments, the expected target engagement level has been previously determined. In some such embodiments, the expected target engagement level has been calculated as the average target engagement level in a group of reference subjects (such as at least three reference subjects, or at least five reference subjects), following administration of a particular dose of anti-ICOS antibody. In some embodiments, the target engagement in the peripheral blood of the reference subjects, such as on T cells, is determined at various time points following administration of the ICOS agonist antibody. The timing of the subsequent dose is then selected based on the average target engagement level in the reference subjects.

### III. Combination Therapy

In some embodiments, an ICOS agonist, such as an ICOS agonist antibody, is administered concurrently with, or sequentially with a second therapeutic agent, such as before or after a second therapeutic agent. For example, in some embodiments, two or more therapeutic agents can be administered sequentially with a time separation of more than about 15, 30, or 60 minutes, 1 day, 2 days, 3 days, 1 week, 2 weeks, 3 weeks, 4 weeks, 6 weeks, or longer. In some embodiments, the ICOS agonist antibody is administered sequentially with a second therapeutic agent with a time separation of 3 weeks. In some embodiments, the ICOS agonist antibody is administered concurrently with a second therapeutic agent, e.g., with one agent immediately following the other.

IV. Combinations with Vaccine Therapies

A study by Thevarajan et al. Nature Medicine (2020) highlighted the developing immune response of a patient who was infected with SARS-CoV-2 and recovered from symptoms of COVID-19, the disease caused by the SARS-CoV-2 virus. ICOS+(or ICOS^{hi}) Tfh emerged in the patient's blood prior to and throughout recovery. Previously, in a SARS-CoV-1 infection model in mice, CD4⁺ T cells were shown to be required to limit the associated lung pathology and for neutralizing antibody production to the virus. ICOS^{hi} cells, which emerge based on antigen stimulation of CD4⁺ cells, represent a potentially universal mechanism of a productive immune response. (Chen et al., J. Virology (2010).) Selectively stimulating ICOS^{hi} cells with an ICOS agonist might allow for the acceleration of, or for a more extensive, humoral response to infectious agent antigens. Thus, stimulating ICOS^{hi} cells with an ICOS agonist may help to increase an immune response in disease scenarios where a productive immune response depends upon, or is influenced by, CD4⁺ cells with high levels of ICOS. Similarly, stimulating ICOS^{hi} cells with an ICOS agonist may help to improve immune responses to vaccines against such infectious diseases.

In some embodiments, an ICOS agonist is administered in combination with a vaccine.

In some embodiments, the vaccine is a vaccine for a bacterial infection or a viral infection. In some embodiments, the vaccine is a vaccine for bacterial vaginosis, Staphylococcus aureus, Pseudomonas aeruginosa, Streptococcus pneumoniae, Corynebacterium diphtheriae (diphtheria), Clostridium tetani (tetanus), Borrelia bacterium (Lyme disease), Clostridium difficile, Bordetella pertussis, human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus. In some embodiments, the vaccine is a vaccine for MERS-CoV, SARS-CoV or SARS-CoV-2. In some embodiments, the vaccine is a vaccine for COVID-19 (i.e., viral disease caused by SARS-CoV-2). In some embodiments, the vaccine is a vaccine for viral disease is caused by an influenza virus. In some embodiments, the influenza virus is influenza virus A, influenza virus B, influenza virus C or influenza virus D. In other embodiments, the vaccine is a vaccine for an infectious disease caused by a pathogenic microorganism, such as bacteria, protozoa, fungi, etc. In some such embodiments, the vaccine is a vaccine for Streptococcus pneumoniae.

Thus, in some embodiments, a method of enhancing the effectiveness of a vaccine is provided, comprising administering one or more doses of an ICOS agonist, such as an ICOS agonist antibody, to the subject and administering one or more doses of a vaccine to the subject. In some embodiments, the ICOS agonist or ICOS agonist antibody is capable of enhancing the effectiveness of the vaccine. For example, the enhanced effectiveness may be demonstrated by clinical comparison between subjects receiving only the vaccine and subjects receiving both the vaccine and the ICOS agonist.

In some embodiments, the ICOS agonist antibody is vopratelimab, BMS-986226, KY1044, KY1055, or GSK3359609, or an antibody comprising the CDR or variable region (VH and VL) sequences of one of those antibodies.

In some embodiments, a method of enhancing the effectiveness of a shingles (varicella zoster) vaccine is provided, comprising administering one or more doses of an ICOS agonist, such as an ICOS agonist antibody, to the subject and administering one or more doses of the vaccine to the subject. Shingles is a reactivation of a latent varicella-zoster virus infection that causes a painful rash. Although shingles can occur anywhere on your body, it most often appears as a single stripe of blisters that wraps around either the left or the right side of your torso. In some embodiments, a shingles vaccine reduces the risk of shingles and/or is administered after shingles symptoms have appeared and shortens the duration of the symptoms and/or complications of shingles. Complications of shingles include postherpetic neuralgia, vision loss, encephalitis, facial paralysis, hearing and balance problems, and skin infections. In some embodiments, administration of an ICOS agonist with a shingles vaccine enhances the effectiveness of the vaccine such that the combination therapy reduces the risk of shingles and/or is administered after shingles symptoms have appeared and shortens the duration of the symptoms and/or complications of shingles to a greater extent than the shingles vaccine alone.

In some embodiments, a subject receiving combination therapy of a shingles vaccine and ICOS agonist is at least 20 years old, at least 30 years old, at least 40 years old, at least 50 years old, or at least 60 years old. In some embodiments, the subject has a weakened immune system, which may increase the changes of developing shingles. A weakened immune system may be caused, in some instances, by certain cancer treatments and/or medications. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In some embodiments, the shingles vaccine is Zostavax (Merck) or Shingrix (GSK). Zostavax is a live attenuated vaccine given in a single dose. Zostavax is recommended for subjects >60 years old or >50 years old with an underlying condition that increases the risk of developing shingles. Zostavax is about 64% effective in subjects aged 60-69, 41% effective in subjects 70-79, and 18% effective in subjects >80 years old. Shingrix is a subunit vaccine given in two doses. Shingrix is recommended for subjects >50 years old, and is 97% effective overall, and 90% effective in subjects >70. Zostavax has fewer side effects than Shingrix, however, so improving the efficacy of Zostavax would be desirable.

In some embodiments, enhancing the effectiveness of a S. Pneumoniae vaccine is provided, comprising administering one or more doses of an ICOS agonist, such as an ICOS agonist antibody, to the subject and administering one or more doses of the vaccine to the subject. S. Pneumoniae infections can cause pneumonia, ear infections, sinus infections, meningitis, and bacteremia. Potential complications of S. Pneumoniae infections include death, brain damage, hearing loss, empyema, pericarditis, lung obstruction, and lung abscess. Risk factors of S. Pneumoniae infections include age (over 65 years), and adults (19 years and older) with chronic illness (such as chronic heart, liver, kidney, or lung disease), diabetes, alcoholism, weakened immune system, cochlear implants, CSF leaks, and smoking. In some embodiments, administration of an ICOS agonist with a S. Pneumoniae vaccine enhances the effectiveness of the vaccine such that the combination therapy reduces the risk of shingles and/or is administered after S. Pneumoniae symptoms have appeared and shortens the duration of the symptoms and/or complications of S. Pneumoniae to a greater extent than the S. Pneumoniae vaccine alone.

In some embodiments, a subject receiving combination therapy of a S. Pneumoniae vaccine and ICOS agonist is at least 20 years old, at least 30 years old, at least 40 years old, at least 50 years old, or at least 60 years old. In some embodiments, the subject is at least 65 years old. In some embodiments, the subject is younger than 65 years old and has a chronic illness (such as chronic heart, liver, kidney, or lung disease), diabetes, alcoholism, weakened immune system, cochlear implants, CSF leaks, and/or is a smoker. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In some embodiments, the S. Pneumoniae vaccine is Prevnar 13 (Pfizer) or PneumoVax 23 (Merck). Prevnar 13 is administered in a four dose schedule in infants and toddlers and is 97% effective. PneumoVax 23 is administered as a single dose, typically in adults 65 years or older, and is 60-70% effective. In some instances, PneumoVax 23 is administered in subjects between 2 and 64 years old with certain medical conditions, or in adults between 19 and 64 years old who smoke cigarettes. PneumoVax 34 is a T-independent vaccine, meaning T cells are not required for vaccine response, but can enhance response by supplying IL-2, IL-3, and/or IFNγ for B cell activation. In some embodiments, coadministration of an ICOS agonist with a T-independent vaccine such as PneumoVax 34, stimulates IFNγ and enhances B cell response to the vaccine.

In some embodiments, enhancing the effectiveness of an influenza vaccine is provided, comprising administering one or more doses of an ICOS agonist, such as an ICOS agonist antibody, to the subject and administering one or more doses of the vaccine to the subject. Influenza is a seasonal respiratory disease in temperate regions, which can cause fever, chills, cough, fore throat, runny nose, congestion, muscle aches, headaches, and fatigue. In some instances, influenza may cause complications such as secondary infections in the respiratory system, myocarditis, and death. Subjects with certain risk factors, such as age (<2 and >64 years old), pregnancy, weakened immune system, asthma, diabetes, and heart disease, may develop more serious disease. In some embodiments, administration of an ICOS agonist with an influenza vaccine enhances the effectiveness of the vaccine such that the combination therapy reduces the risk of shingles and/or is administered after influenza symptoms have appeared and shortens the duration of the symptoms and/or complications of influenza to a greater extent than the influenza vaccine alone.

In some embodiments, an ICOS agonist is administered in combination with a seasonal inactivated influenza vaccine, which includes split egg-based vaccines, such as quadrivalent vaccines (6 months+, Sanofi, Seqirus, GSK), quadrivalent ID low dose vaccines (18-64 years old, Sanofi), trivalent high dose vaccine (>64 years old, Sanofi), and trivalend adjuvanted (MF59) (>64 years old, Seqirus); and cell-based split vaccines such as Flucellvax (4 years old+; Seqirus). In some embodiments, an ICOS agonist is administered in combination with a recombinant seasonal influenza vaccine, such as an insect cell purified vaccine including quadrivalent FluBlok (>18 years, Sanofi). In some embodiments, an ICOS agonist is administered in combination with a live attenuated seasonal influenza vaccine, such as an egg-based vaccine such as quadrivalend FluMist (2-49 years old, AstraZeneca). Generally, seasonal influenza vaccines are less effective in subjects 65 years old or older, with effectiveness rates in the range of about 35% to 45%.

In some embodiments, an ICOS agonist is administered in combination with a pandemic influenza vaccine, such as the H5N1 vaccines (Sanofi; GSK; Seqirus). Pandemic influenza vaccines have effectiveness rates in the range of about 44% to about 95%. As with seasonal influenza vaccines, pandemic influenza vaccines are less effective in elderly subjects.

In some embodiments, administering an ICOS agonist in combination with a seasonal influenza vaccine or pandemic influenza vaccine increases effectiveness of the vaccine. In some embodiments, a subject receiving combination therapy of an influenza vaccine and ICOS agonist is at least 20 years old, at least 30 years old, at least 40 years old, at least 50 years old, or at least 60 years old. In some embodiments, the subject is at least 65 years old. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In some embodiments, enhancing the effectiveness of a coronavirus vaccine is provided, comprising administering one or more doses of an ICOS agonist, such as an ICOS agonist antibody, to the subject and administering one or more doses of the vaccine to the subject. In some embodiments, the coronavirus vaccine is a vaccine for COVID-19.

In some embodiments, a combination therapy comprising an ICOS agonist and a vaccine is administered after a subject has exhibited one or more symptoms of the disease. In some embodiments, the subject to be treated has a viral disease. Exemplary types of viral disease include, but are not limited to viral disease caused by human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus. In some embodiments, the viral disease is caused by a coronavirus. In some embodiments, the coronavirus is MERS-CoV, SARS-CoV or SARS-CoV-2. In some embodiments, the disease is COVID-19 (i.e., viral disease caused by SARS-CoV-2). In some embodiments, the viral disease is caused by an influenza virus. In some embodiments, the influenza virus is influenza virus A, influenza virus B, influenza virus C or influenza virus D. In other embodiments, the infectious disease is caused by a pathogenic microorganism, such as bacteria, protozoa, fungi, etc. Nonlimiting exemplary bacterial diseases include diseases caused by Staphylococcus aureus, Pseudomonas aeruginosa, Streptococcus pneumoniae, Corynebacterium diphtheriae (diphtheria), Clostridium tetani (tetanus), Borrelia bacterium (Lyme disease), Clostridium difficile, and Bordetella pertussis.

In some embodiments, the subject has a viral disease, or is at risk of developing a viral disease, caused by SARS-CoV-2. In some embodiments, the subject has tested positive for SARS-CoV2. In some embodiments, the subject has exhibited symptoms of SARS-CoV-2 infection. In some embodiments, the subject has not exhibited symptoms of SARS-CoV-2 infection prior to administration of ICOS agonist antibody. Exemplary symptoms include, for example, fever, coughing, shortness of breath, fatigue, body aches, digestive upset, and/or loss of sense of smell and/or taste. In some embodiments, the subject has symptoms that comprise coughing, fever and/or shortness of breath. In some embodiments, the subject to be treated is at least 20 years old, at least 30 years old, at least 40 years old, at least 50 years old, at least 60 years old, at least 65 years old, at least 70 years old, at least 75 years old, or at least 80 years old. In some embodiments, the subject has one ore more underlying health conditions, such as obesity, diabetes, smoking, chronic illness (such as chronic heart, liver, kidney, or lung disease), or a weakened immune system. In some embodiments, the subject is at least 60 years old. In some embodiments, the subject has or has been diagnosed with an immune disorder selected from Wiskott-Aldrich syndrome, X-linked thrombocytopenia (XLT), CVID, hypogammaglobulinemia, and/or idiopathic CD4 lymphopenia.

In some embodiments, the vaccine and ICOS agonist can be administered concurrently or sequentially, such as with a time separation of more than about 15, 30, or 60 minutes, 1 day, 2 days, 3 days, 1 week, 2 weeks, 3 weeks, 4 weeks, 6 weeks, or longer. In some embodiments, the ICOS agonist antibody is administered sequentially with a second therapeutic agent with a time separation of 3 weeks. In some embodiments, the ICOS agonist antibody is administered concurrently with a second therapeutic agent, e.g., with one agent immediately following the other. In some embodiments, the method comprises administering an ICOS agonist or ICOS agonist antibody to a subject after the subject has been administered the vaccine. In some embodiments, the subject has received a complete dose of vaccine prior to administration of the ICOS agonist or ICOS agonist antibody.

In some embodiments, the method comprises administering one or more doses of the ICOS agonist antibody. In some embodiments, the method comprises administering at least one, at least two, at least three, at least four, or at least five doses of the ICOS agonist antibody after the last dose of the vaccine has been administered. In some embodiments, the first dose of the ICOS agonist antibody is administered after the first dose of the vaccine. In some embodiments, the first dose of the ICOS agonist antibody is administered three weeks after the first dose of the vaccine. In some embodiments, the ICOS agonist antibody is administered once. In some embodiments, the ICOS agonist antibody is administered more than once and is administered once every three weeks. In some embodiments, the ICOS agonist antibody is administered at the same time as the vaccine.

In some embodiments, cells within a sample of the infected cells express ICOS. In some such embodiments, the infected cells may be considered to be ICOS-positive, ICOS⁺, ICOS^{Hi}, or to express ICOS. Expression of ICOS may be determined by, for example, by IHC.

In some embodiments, the ICOS agonist antibody is provided at a dose of, for example, 0.01 mg/kg, 0.03 mg/kg, 0.1 mg/kg or 0.3 mg/kg. In some embodiments, the therapeutically effective amount is from 0.03 mg/kg to 0.3 mg/kg. In some embodiments, the therapeutically effective amount is from 0.03 mg/kg to 0.1 mg/kg. In some embodiments, the therapeutically effective amount is between 0.1 mg/kg and 0.3 mg/kg. In some embodiments, the therapeutically effective amount is 0.1 mg/kg. In some embodiments, the therapeutically effective amount is 0.03 mg/kg. In some embodiments, the therapeutically effective amount is 0.03, 0.04, 0.05, 0.06, 0.07. 0.08, 0.09, or 0.1 mg/kg.

The ICOS agonist may also be administered in an amount effective for increasing the number of Teff cells; activating Teff cells; depleting Treg cells; and/or increasing the ratio of Teff cells to Treg cells. In some embodiments, the Treg cells are CD4⁺ FoxP3+ T cells. In some embodiments, the Teff cells are CD8+ T cells. In some embodiments, the Teff cells are CD4+ FoxP3+ T cells and CD8+ T cells. In some embodiments, the Teff cells are CD4+ FoxP3-T cells.

In some embodiments, treatment with ICOS agonist antibody results in a pharmacodynamics readout, as described herein.

### V. Pharmaceutical Compositions

In some embodiments, compositions comprising ICOS agonists, and/or compositions comprising anti-infectious disease therapies are provided herein. In some embodiments, the ICOS agonist can be formulated with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof. In some embodiments, the pharmaceutical composition comprises a humanized antibody. In some embodiments, the pharmaceutical composition comprises an antibody prepared in a host cell or cell-free system. In some embodiments, the pharmaceutical composition comprises pharmaceutically acceptable carrier.

In some embodiments, pharmaceutical compositions are administered in an amount effective for treatment of infectious diseases, such as viral diseases. The therapeutically effective amount is typically dependent on the weight of the subject being treated, his or her physical or health condition, the extensiveness of the condition to be treated, or the age of the subject being treated. In some embodiments, pharmaceutical compositions are administered in an amount effective for enhancing the effectiveness of a vaccine against an infectious disease, such as a viral disease.

In some embodiments, ICOS agonists can be administered in vivo by various routes, including, but not limited to, intravenous, intra-arterial, parenteral, intratumoral, intraperitoneal or subcutaneous. The appropriate formulation and route of administration may be selected according to the intended application.

In some embodiments, a pharmaceutical composition comprises a vaccine in combination with an ICOS agonist.

### VI. Exemplary ICOS Agonist Antibodies

In some embodiments, the ICOS agonist used herein is an ICOS agonist antibody. ICOS agonist antibodies include, but are not limited to, humanized antibodies, chimeric antibodies, mouse antibodies, human antibodies, and antibodies comprising the heavy chain and/or light chain CDRs discussed herein. In some embodiments, an isolated antibody that binds to ICOS is provided. In some embodiments, a monoclonal antibody that binds to ICOS is provided. In some embodiments, the ICOS agonist antibody is an ICOS agonist antibody. In some embodiments, administration of the ICOS agonist antibodies described herein increases the number of Teff cells and/or activates Teff cells and/or decreases Treg cells in a subject; and/or increases the ratio of Teff cells to Treg cells. In some embodiments, the Treg cells are CD4+ FoxP3+ T cells. In some embodiments, the Teff cells are CD8+ T cells. In some embodiments, the Teff cells are CD4+ FoxP3− T cells and CD8+ T cells.

ICOS agonist antibodies described herein may be expressed and produced as described in WO 2016/154177 and WO 2017/070423, the content of each of which is incorporated by reference. See WO 2016/154177 and WO 2017/070423, which are each specifically incorporated herein by reference. Exemplary therapeutic ICOS agonist antibodies include, but are not limited to, JTX-2011 (vopratelimab, Jounce Therapeutics; US 2016/0304610; WO 2016/154177; WO 2017/070423); GSK-3359069 (GSK); KY1044 (Kymab); KY1055 (Kymab); and BMS-986226 (Bristol-Myers Squibb), and antibodies comprising the CDRs or variable regions (VH and VL) of one of those antibodies. In certain embodiments, the ICOS agonist antibody is an antibody having light and heavy chain sequences corresponding to SEQ ID NOs: 16 and 15, respectively; or SEQ ID NOs: 16 and 17, respectively). In some embodiments, the ICOS agonist antibody is vopratelimab.

In some embodiments, the ICOS agonist antibody comprises six CDRs including (a) HCDR1 comprising the amino acid sequence of SEQ ID NO: 9; (b) HCDR2 comprising the amino acid sequence of SEQ ID NO: 10; (c) HCDR3 comprising the amino acid sequence of SEQ ID NO: 11; (d) LCDR1 comprising the amino acid sequence of SEQ ID NO: 12; (e) LCDR2 comprising the amino acid sequence of SEQ ID NO: 13; and (f) LCDR3 comprising the amino acid sequence of SEQ ID NO: 14.

In some embodiments, an ICOS agonist antibody comprises a heavy chain variable region and a light chain variable region. In some embodiments, a therapeutic ICOS agonist antibody comprises at least one heavy chain comprising a heavy chain variable region and at least a portion of a heavy chain constant region, and at least one light chain comprising a light chain variable region and at least a portion of a light chain constant region. In some embodiments, a therapeutic ICOS agonist antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some embodiments, the heavy chain is the region of the ICOS agonist antibody that comprises the three heavy chain CDRs. In some embodiments, the light chain is the region of the therapeutic ICOS agonist antibody that comprises the three light chain CDRs.

In some embodiments, an ICOS agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 7. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, but an ICOS agonist antibody comprising that sequence retains the ability to bind to ICOS. In some embodiments, a total of 1 to 10 amino acids have been substituted, inserted and/or deleted in SEQ ID NO: 7. In some embodiments, substitutions, insertions, or deletions occur in regions outside the CDRs (that is, in the FRs). Optionally, the ICOS agonist antibody comprises the VH sequence in SEQ ID NO: 7, including post-translational modifications of that sequence.

In some embodiments, the VH comprises: (a) HCDR1 comprising the amino acid sequence of SEQ ID NO: 9; (b) HCDR2 comprising the amino acid sequence of SEQ ID NO: 10; and (c) HCDR3 comprising the amino acid sequence of SEQ ID NO: 11.

In some embodiments, an ICOS agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 8. In some embodiments, a VL sequence having at least 90%, 91%, 92%, 93%, 94%, 95%, 96%, 97%, 98%, or 99% identity contains substitutions (for example, conservative substitutions), insertions, or deletions relative to the reference sequence, but an ICOS agonist antibody comprising that sequence retains the ability to bind to ICOS. In some embodiments, a total of 1 to 10 amino acids have been substituted, inserted and/or deleted in SEQ ID NO: 8. In some embodiments, the substitutions, insertions, or deletions occur in regions outside the CDRs (that is, in the FRs). Optionally, the ICOS agonist antibody comprises the VL sequence in SEQ ID 8, including post-translational modifications of that sequence.

In some embodiments, the VL comprises: (a) LCDR1 comprising the amino acid sequence of SEQ ID NO: 12; (b) LCDR2 comprising the amino acid sequence of SEQ ID NO: 13; and (c) LCDR3 comprising the amino acid sequence of SEQ ID NO: 14.

In some embodiments, an ICOS agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 7, and a light chain variable domain (VL) having at least 90%, 91%, 92%, 93%, 94%, 95%, 96%, 97%, 98%, 99%, or 100% sequence identity to the amino acid sequence of SEQ ID NO: 8. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an ICOS agonist antibody comprising that sequence retains the ability to bind to ICOS. In some embodiments, a total of 1 to 10 amino acids have been substituted, inserted and/or deleted in SEQ ID NO: 7. In some embodiments, a total of 1 to 10 amino acids have been substituted, inserted and/or deleted in SEQ ID NO: 8. In some embodiments, substitutions, insertions, or deletions occur in regions outside the CDRs (that is, in the FRs). Optionally, the ICOS agonist antibody comprises the VH sequence in SEQ ID NO: 7, and the VL sequence of SEQ ID NO: 8, including post-translational modifications of one or both sequence.

In some embodiments, the ICOS agonist antibody comprises (I) a VH domain comprising: (a) HCDR1 comprising the amino acid sequence of SEQ ID NO: 9; (b) HCDR2 comprising the amino acid sequence of SEQ ID NO: 10; and (c) HCDR3 comprising the amino acid sequence of SEQ ID NO: 11; and (II) a VL domain comprising: (d) LCDR1 comprising the amino acid sequence of SEQ ID NO: 12; (e) LCDR2 comprising the amino acid sequence of SEQ ID NO: 13; and (f) LCDR3 comprising the amino acid sequence of SEQ ID NO: 14.

In some embodiments, the ICOS agonist antibody comprises the VH and VL sequences in SEQ ID NO: 7 and SEQ ID NO: 8, respectively, including post-translational modifications of those sequences.

In some embodiments, the ICOS agonist antibody binds to ICOS and increases the number of Teff cells and/or activates Teff cells and/or decreases the number of Treg cells and/or increases the ratio of Teff cells to Treg cells in a mammal, such as a human. In some embodiments, the Treg cells are CD4+ FoxP3+ T cells. In some embodiments, the Teff cells are CD8+ T cells. In some embodiments, the Teff cells are CD4+ FoxP3− T cells and/or CD8+ T cells.

### VII. Antibody Expression and Production

a. Nucleic Acid Molecules Encoding Antibodies

Nucleic acid molecules comprising polynucleotides that encode one or more chains of an antibody are provided herein. In some embodiments, a nucleic acid molecule comprises a polynucleotide that encodes a heavy chain or a light chain of an antibody. In some embodiments, a nucleic acid molecule comprises both a polynucleotide that encodes a heavy chain and a polynucleotide that encodes a light chain, of an ICOS agonist antibody. In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, the nucleic acid is one that encodes for any of the amino acid sequences for the antibodies in the Sequence Table herein.

Nucleic acid molecules can be constructed using recombinant DNA techniques conventional in the art. In some embodiments, a nucleic acid molecule is an expression vector that is suitable for expression in a selected host cell. Vectors

Vectors comprising polynucleotides that encode heavy chains and/or light chains are provided. Vectors comprising polynucleotides that encode heavy chains and/or light chains are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

b. Host Cells

In some embodiments, antibody heavy chains and/or antibody light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO—S, DG44. Lecl3 CHO cells, and FUT8 CHO cells; PER.C6® cells (Crucell); and NSO cells. In some embodiments, ICOS agonist antibody heavy chains and/or ICOS agonist antibody light chains may be expressed in yeast. See, for example, U.S. Publication No. US 2006/0270045 A1. In some embodiments, a particular eukaryotic host cell is selected based on its ability to make desired post-translational modifications to the ICOS agonist antibody heavy chains and/or ICOS agonist antibody light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the polynucleotides or vectors described herein are also provided. In some embodiments, a host cell comprising an antibody is provided. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

c. Purification of Antibodies

Antibodies can be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an antibody. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

d. Cell-Free Production of Antibodies

In some embodiments, an antibody is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

e. Antibody Compositions

In some embodiments, antibodies prepared by the methods described above are provided. In some embodiments, the antibody is prepared in a host cell. In some embodiments, the antibody is prepared in a cell-free system. In some embodiments, the antibody is purified. In some embodiments, the antibody prepared in a host cell or a cell-free system is a chimeric antibody. In some embodiments, the antibody prepared in a host cell or a cell-free system is a humanized antibody. In some embodiments, the antibody prepared in a host cell or a cell-free system is a human antibody. In some embodiments, a cell culture media comprising an antibody is provided. In some embodiments, a host cell culture fluid comprising an antibody is provided.

In some embodiments, compositions comprising antibodies prepared by the methods described above are provided. In some embodiments, the composition comprises an antibody prepared in a host cell. In some embodiments, the composition comprises an antibody prepared in a cell-free system. In some embodiments, the composition comprises a purified antibody. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

In some embodiments, a composition comprising an antibody at a concentration of more than about any one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL is provided. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

VIII. Exemplary Methods for Detection of ICOS Expression and Target Engagement

Provided herein are methods of assessing patient responsiveness to one or more infectious disease treatments. In some embodiments, methods of identifying a subject who may benefit from continued treatment with one or more infectious disease therapies, optionally in combination with an ICOS agonist antibody, are provided.

In some embodiments, the infectious disease treatment comprises an ICOS agonist antibody. In some embodiments, the antibody is vopratelimab, BMS-986226, KY1044, KY1055, or GSK3359609, or an antibody comprising the CDR or variable domain sequences of one of those antibodies.

In some embodiments, the subject to be treated has a viral disease. Exemplary types of viral disease include, but are not limited to viral disease caused by human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus. In some embodiments, the viral disease is caused by a coronavirus. In some embodiments, the coronavirus is MERS-CoV, SARS-CoV or SARS-CoV-2. In some embodiments, the disease is COVID-19 (i.e., viral disease caused by SARS-CoV-2). In some embodiments, the viral disease is caused by an influenza virus. In some embodiments, the influenza virus is influenza virus A, influenza virus B, influenza virus C or influenza virus D. In other embodiments, the infectious disease is caused by a pathogenic microorganism, such as bacteria, protozoa, fungi, etc. Nonlimiting exemplary bacterial diseases include diseases caused by Staphylococcus aureus, Pseudomonas aeruginosa, Streptococcus pneumoniae, Corynebacterium diphtheriae (diphtheria), Clostridium tetani (tetanus), Borrelia bacterium (Lyme disease), Clostridium difficile, and Bordetella pertussis.

In some embodiments, the subject has a viral disease caused by SARS-CoV-2. In some embodiments, the subject has tested positive for SARS-CoV2. In some embodiments, the subject has exhibited symptoms of SARS-CoV-2 infection. In some embodiments, the subject has not exhibited symptoms of SARS-CoV-2 infection prior to administration of ICOS agonist antibody. Exemplary symptoms include, for example, fever, coughing, shortness of breath, fatigue, body aches, digestive upset, and/or loss of sense of smell and/or taste. In some embodiments, the subject has symptoms that comprise coughing, fever and/or shortness of breath. In some embodiments, the subject to be treated is at least 60 years old, at least 65 years old, at least 70 years old, at least 75 years old, or at least 80 years old.

a. Exemplary Antibody-Based Detection Methods

In some embodiments, the methods include determining whether a patient treated has CD4+ T cells in peripheral blood that have elevated expression of ICOS using, for example, an ICOS detection antibody. In some embodiments, the methods of detection include contacting a patient sample (e.g., a peripheral blood sample, or a fraction thereof) with an antibody, and determining whether the level of binding differs from that of a control. In some embodiments, CD4+ T cells from the peripheral blood test sample are contacted with an ICOS detection antibody and binding between the antibody and the CD4⁺ T cells is determined. When CD4+ T cells from a test sample are shown to have an increase in binding activity to the antibody, as compared to CD4+ T cells from a control sample, continued treatment with a therapy comprising an ICOS agonist antibody treatment, as described herein.

Various methods known in the art for detecting specific antibody-antigen binding can be used. These assays include, but are not limited to, flow cytometry (including, for example, fluorescent activating cell sorting (FACS)), indirect immune-fluorescence, solid phase enzyme-linked immunosorbent assay (ELISA), ELISpot assays, fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA), western blotting (including in cell western), immunofluorescent staining, microengraving (see Han et al., Lab Chip 10(11):1391-1400, 2010), Quant-iT and Qubit protein assay kits, NanoOrange protein quantitation kit, CBQCA protein quantitation kits, EZQ protein quantitation kit, Click-iT reagents, Pro-Q Diamond phosphoprotein stain, Pro-Q glycoprotein stain kits, peptide and protein sequencing, N-terminal amino acid analysis (LifeScience Technologies, Grand Island, N.Y.), chemiluminescence or colorimetric based ELISA cytokine Arrays (Signosis) Intracellular Cytokine Staining (ICS), BD Phosflow™ and BD™ Cytometric Bead Arrays (BD Sciences, San Jose, Calif.); CyTOF Mass Cytometer (DVS Sciences, Sunnyvale Calif.); Mass Spectrometry, Microplate capture and detection assay (Thermo Scientific, Rockland, IL), Multiplex Technologies (for example Luminex, Austin, Tex.); FlowCellect™ T-cell Activation Kit (EMD Millipore); Surface Plasmon Resonance (SPR)-based technologies (for example Biacore, GE Healthcare Life Sciences, Uppsala, Sweden); CD4+ Effector Memory T-cell Isolation Kit and CD8+ CD45RA+ Effector T-cell Isolation Kit (Miltenyi Biotec Inc., CA); The EasySep™ Human T-cell Enrichment Kit (StemCells, Inc., Vancouver, Canada); HumanTh1/Th2/Th17 Phenotyping Kit (BD Biosciences, CA); immunofluorescent staining of incorporated bromodeoxyuridine (BrdU) or 7-aminoactinomycin D. See also, Current Protocols in Immunology (2004) sections 3.12.1-3.12.20 by John Wiley & Sons, Inc., or Current Protocols in Immunology (2013) or by John Wiley & Sons, Inc., the contents of which are herein incorporated by reference in their entirety.

An indicator moiety, or label group, can be attached to the subject antibodies and is selected so as to meet the needs of various uses of the method which are often dictated by the availability of assay equipment and compatible immunoassay procedures.

Appropriate labels include, without limitation, radionuclides (for example 125I, 131I, 35S, 3H, or 32P), enzymes (for example, alkaline phosphatase, horseradish peroxidase, luciferase, or β-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif). General techniques to be used in performing the various immunoassays noted above are known to those of ordinary skill in the art.

In some instances, the ICOS detection antibodies need not be labeled, and the presence thereof can be detected using a second labeled antibody which binds to the first ICOS agonist antibody.

In some embodiments, the CD4⁺ cells from the peripheral blood test sample are contacted with an ICOS detection antibody and the binding between the antibody and the CD4⁺ cells is determined. In some embodiments, the level of total ICOS expression in CD4⁺ T cells is determined using a fluorescence activated cell sorter. Fluorescence activated cell sorters can have varying degrees of sophistication, such as multiple color channels, low angle and obtuse light scattering detecting channels, impedance channels, etc. Cells may be selected against dead cells by employing dyes associated with dead cells (e.g., propidium iodide). The FACS apparatus commonly includes a light source, usually a laser, and several detectors for the detection of cell particles or subpopulations of cells in a mixture using light scatter or light emission parameters. The underlying mechanisms of FACS are well known in the art, and essentially involve scanning (e.g., counting, sorting by size or fluorescent label) single particles are they flow in a liquid medium past an excitation light source. Light is scattered and fluorescence is emitted as light from the excitation source strikes the moving particle. Forward scatter (FSC, light scattered in the forward direction, i.e., the same direction as the beam) provides basic morphological information about the particles, such as cell size and morphology. Light that is scattered at 90° to the incident beam is due to refracted or reflected light, and is referred to as side angle scatter (SSC). This parameter measures the granularity and cell surface topology of the particles. Collectively, scatter signals in both the forward and wide angle direction are used to identify subpopulations of cells based on cell size, morphology, and granularity. This information is used to distinguish various cellular populations in a heterogeneous sample.

Exemplary ICOS antibodies for use in the detection aspects of the methods described herein include antibodies that recognize an internal (i.e., intracellular) epitope of ICOS. While ICOS can be expressed on the surface of T cells, it is estimated that a large proportion of total cellular ICOS (e.g., about 80%) is present in intracellular stores. While exemplary therapeutic ICOS agonist antibodies, such as vopratelimab, recognize extracellular epitopes of ICOS, the use of an ICOS detection antibody that specifically binds to an intracellular ICOS epitope allows for the determination of total ICOS expression levels. Examples of antibodies that recognize intracellular ICOS epitopes, and thus which can be used in methods to detect total ICOS, include 2M13 and 2M19 (see WO 2017/070423), and variants thereof. In addition, antibodies that compete with 2M13 and 2M19 for binding to ICOS can be used to detect ICOS according to the methods of the invention.

In some embodiments, target engagement is measured following administration of an ICOS agonist antibody described herein. In some such embodiments, target engagement is measured in peripheral blood, for example, on T cells. Target engagement may be measured using, for example, an anti-ICOS antibody that binds to the same or overlapping epitope of ICOS as the administered therapeutic antibody, so the presence of the therapeutic antibody bound to ICOS blocks binding of the antibody used to measure target engagement.

In some embodiments, target engagement is measured on live CD4⁺ T cells in a sample taken from a subject that has been administered an ICOS agonist antibody. In some such embodiments, a labeled version of the treatment antibody (such as, for example, vopratelimab-DyLight 650) is used to detect free ICOS. Receptor availability may be determined using the following formula (where V stands for vopratelimab):

$$\% Receptor Available at time t = \frac{\begin{matrix}( MFI of VmG 2 a - mG 2 aDy 650 at time t - \\ MFI of isotypeDy 650 at time t )\end{matrix}}{\begin{matrix}( MFI of VmG 2 a - mG 2 aDy 650 prestudy - \\ MFI of isotypeDy 650 prestudy )\end{matrix}} \times 100$$

Target engagement is determined as 100%−% Receptor availability.

In some embodiments, expected target engagement is determined by measuring the target engagement in a group of subjects, such as at least three subject or at least five subjects, following administration of a particular dose of ICOS agonist antibody. In some embodiments, the expected target engagement is an average of the target engagement in the group of subjects after a particular period of time. Expected target engagement may be used to determine the appropriate dosing interval for an ICOS agonist antibody.

b. Exemplary Nucleic Acid-Based Detection Methods

In some embodiments, the methods provided herein include measuring an mRNA level. In some embodiments, the methods provided herein comprise measuring an ICOS mRNA.

Any suitable method of determining mRNA levels may be used. Methods for the evaluation of mRNAs include, for example, hybridization assays using complementary DNA probes (such as in situ hybridization using labeled riboprobes specific for target sequences, Northern blot and related techniques) and various nucleic acid amplification assays (such as RT-PCR using complementary primers specific for target sequences and other amplification type detection methods, such as, for example, branched DNA, SISB A, TMA and the like).

In some embodiments, the mRNA level is determined by quantitative RT-PCR. In some embodiments, the mRNA level is determined by digital PCR. In some embodiments, the mRNA level is determined by RNA-Seq. In some embodiments, the mRNA level is determined by RNase Protection Assay (RPA). In some embodiments, the mRNA level is determined by Northern blot. In some embodiments, the mRNA level is determined by in situ hybridization (ISH). In some embodiments, the mRNA level is determined by a method selected from quantitative RT-PCR, microarray, digital PCR, RNA-Seq, RNase Protection Assay (RPA), Northern blot, and in situ hybridization (ISH).

In some embodiments, for example when quantitative RT-PCR is used, the threshold cycle number is compared between two mRNAs, and the lower threshold indicates a higher level of the respective mRNA. As a non-limiting example, in some embodiments, if levels of ICOS mRNA and at least one reference mRNA are analyzed and the threshold cycle number (Ct) for ICOS is 28 and the Ct for the reference mRNA is 30, then ICOS is at a higher level compared to the reference. In various embodiments, similar comparisons may be carried out for any type of quantitative or semi-quantitative analytical method.

In some embodiments, the level of at least one mRNA is normalized. In some embodiments, the level of at least two mRNAs are normalized and compared to each other. In some embodiments, such normalization may allow comparison of mRNA levels when the levels are not determined simultaneously and/or in the same assay reaction. One skilled in the art can select a suitable basis for normalization, such as at least one reference mRNA or other factor, depending on the assay.

In some embodiments, the at least one reference mRNA comprises a housekeeping gene. In some embodiments, the at least one reference mRNA comprises one or more of RPLP0, PPIA, TUBB, ACTB, YMHAZ, B2M, UBC, TBP, GUSB, HPRT1, or GAPDH.

### IX. EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Profiling of ICOS Hi CD4 T Cells from Cancer Patients

Oligoclonal Population of Effector, Memory, and Tfh-associated Phenotypes

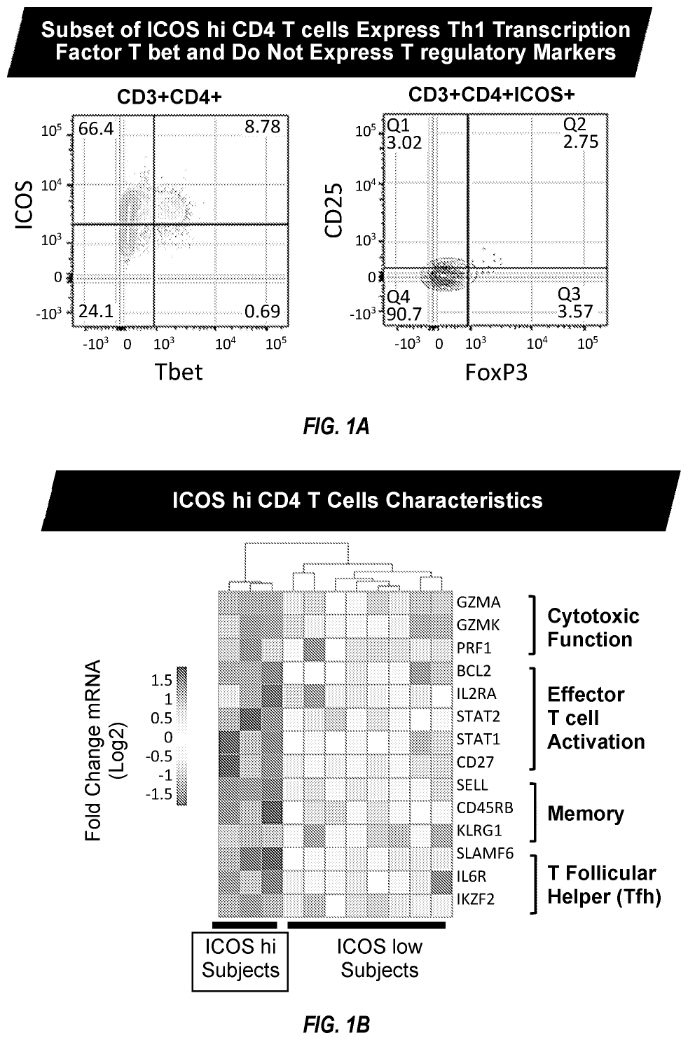

A population of ICOS hi CD4 T cells (ICOS^{hi}CD4⁺) from cancer patients was analyzed for effector, memory and Tfh-associated phenotypes. As shown in FIG. 1A, a subset of ICOS^{hi}CD4⁺ cells express the Th1 transcription factor Tbet. Cells in this subset do not express the T regulatory markers CD25 and FoxP3. In FIG. 1B, the change in mRNA levels for T cell markers was measured in the ICOS^{hi}CD4⁺ population and ICOS low CD4 T cell (ICOS^{lo}CD4⁺) population. The T cell markers were GZMA, GZMK, PRF1, BCL2, IL2RA, STAT2, CD27, SELL, CD45RB, KLRG1, SLAMF6, IL6R and IKZF2. In general, a log 2 fold increase of 0 to 1.5 was observed in the T cell marker mRNAs in ICOS^{high}CD4⁺ subjects while a log 2 fold decrease of 0 to 1.5 was observed in the ICOS^{lo}CD4⁺ subjects.

### Example 2. Vopratelimab Stimulates ICOS hi CD4 T Cells After Sub-Optimal TCR Signal

Polyclonal TCR Stimulation by Sub-Optimal OKT3 at concentrations that are sufficient to drive functional activity in the absence of co-stimulation

In this Example, human CD4⁺ cells were isolated from peripheral blood mononuclear cells (PBMCs) from healthy human donors (Research Blood Components) using Ficoll (GE Life Sciences) centrifugation, frozen in BamBanker (Wako-Chem) and stored at −150° C. until use. Cells were CFSE labeled and then activated with vopratelimab at the 133.4. 66.7, 33.4, 16.7, 8.3, 4.2, 2.1, 1.0, 0.5 and 0.3 nM alone or in combination with suboptimal OKT3 (the anti-human CD3 antibody, Biolegend's OKT3; 1 μg/ml). Soluble anti-CD28 antibody was used as a positive control antibody. After 3 days, the percentage of proliferation was determined using flow cytometry. Proliferation is plotted as the percentage of divided cells (measured by CFSE dilution using flow cytometry) and are means of duplicates.

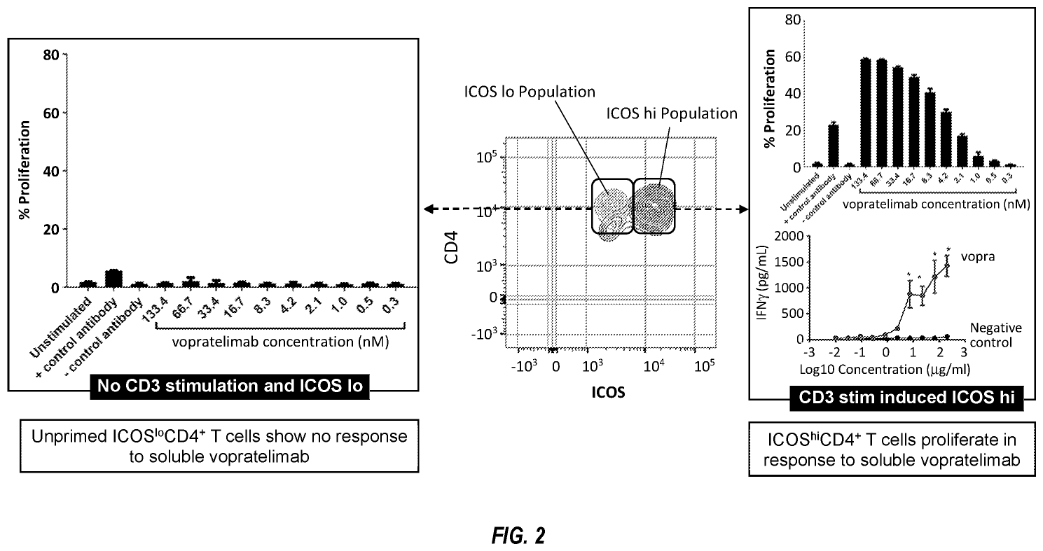

As shown in FIG. 2, middle panel, CD4 T cells from the PBMCs without the OKT3-mediated TCR stimulation displayed an ICOSlo CD4 phenotype whereas the addition of OKT3 induced an ICOShi CD4⁺ T cell phenotype. The left panel shows the effect of increasing concentrations of soluble vopratelimab on change in percent proliferation in ICOS^{lo}CD4⁺ cells. As controls, ICOS^{lo}CD4⁺ cells were unstimulated, treated with a positive control antibody, or treated with a negative control antibody. Unprimed ICOS^{lo}CD4⁺ cells showed no response to vopratelimab. The right panel shows the effect of the same vopratelimab concentrations and controls on percent proliferation in ICOS^{hi}CD4⁺ cells that were induced by TCR priming. ICOS^{hi}CD4⁺ cells showed increased percent proliferation a dose response manner. IFNγ expression levels (pg/ml) also increased with vopratelimab treatment.

### Example 3. Vopratelimab Stimulates Antigen-Specific ICOS Hi CD4 T Cells

### An Ex Vivo Tetanus Toxoid Recall Assay

Peripheral blood mononuclear cells (PBMCs) from three healthy donors were stimulated with soluble recall antigen (tetanus antigen) for 24 hours to induce differentiated ICOS^{hi}and ICOS^{low}CD4 T cell populations (ICOS^{hi}CD4⁺ and ICOS^{lo}CD4⁺ populations). Following stimulation, soluble vopratelimab or isotype control antibody were added to the PBMC solution along with brefeldin A and incubated for 6 hours. Following the 6-hour ex vivo treatment, expression of intracellular cytokine (IFNγ, TNFα, IL-2) was measured by flow cytometry in CD4⁺ICOS^{hi} and CD4⁺ICOS^{lo} populations. Mean levels of cytokines IFN-γ, TNFα, and IL-6 were determined and compared to control populations not treated with soluble vopratelimab.

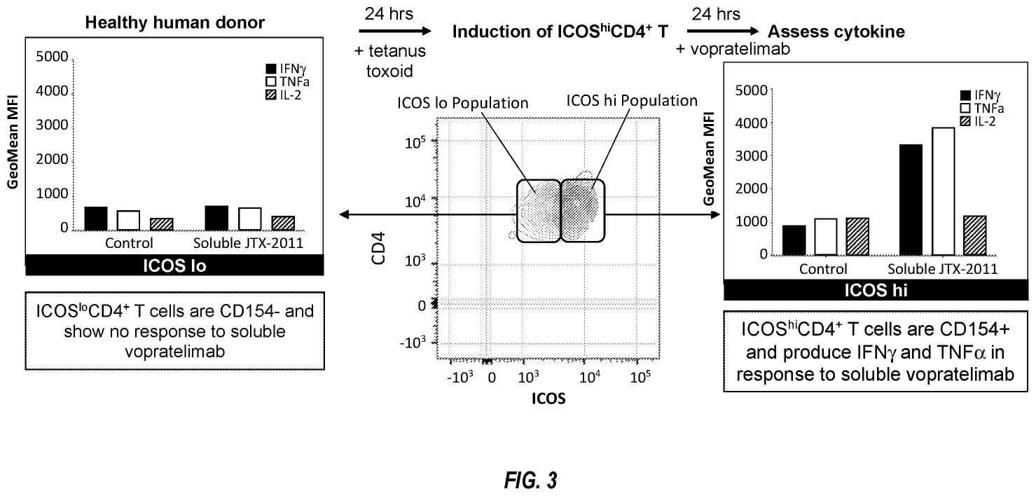

FIG. 3 shows exemplary results using PBMCs from one donor. A significant increase in IFNγ and TNFα expression levels was observed in the ICOS^{hi}CD4⁺ populations following treatment with vopratelimab as compared to treatment with control antibody (right panel); whereas in the ICOS^{lo}CD4⁺ populations, cytokine expression did not increase following treatment with either antibody (left panel). These results demonstrate that treatment of ICOS^{hi}CD4⁺ cells with vopratelimab further enhances IFNγ and TNFα expression, indicating activation of T cells, which in turn cause proliferation of memory B cells.

### Example 4. Vopratelimab Stimulates Antigen-Specific ICOS Hi CD4 T Cells

### An Ex Vivo CMV Recall Assay

The ex vivo CMV recall assay was carried out as described above in Example 3. In this example, PBMCs from three healthy donors were stimulated with PepTivator CMV pp65 peptide (Miltenyi Biotech) instead of the tetanus antigen. Again, mean levels of cytokines IFN-γ, TNFα, and IL-6 were determined and compared to control populations not treated with soluble vopratelimab.

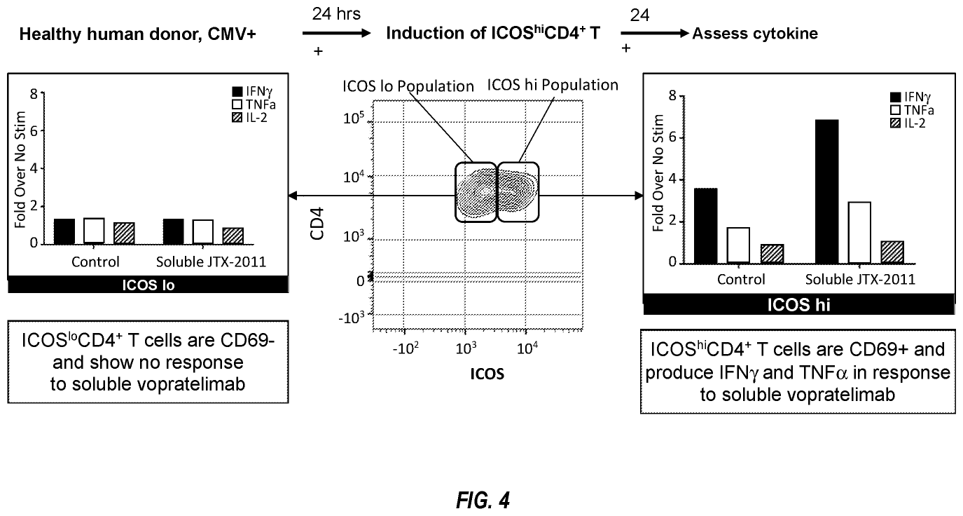

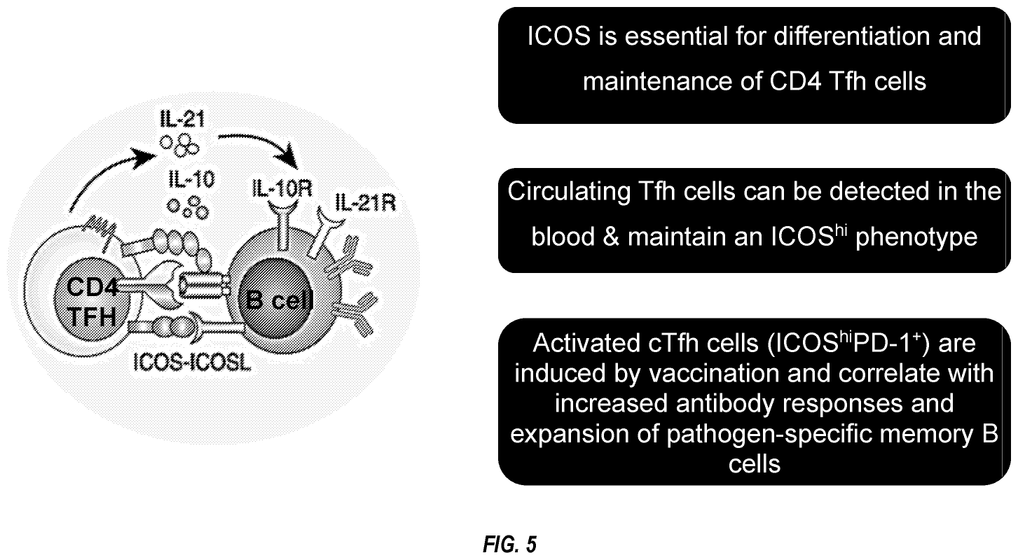

FIG. 4 shows exemplary results using PBMCs from one donor. A significant increase in IFNγ and TNFα expression levels was observed in the ICOS^{hi}CD4⁺ populations following treatment with vopratelimab as compared to treatment with control antibody (right panel); whereas in the ICOS^{lo}CD4⁺ populations, cytokine expression did not increase following treatment with either antibody (left panel). These results demonstrate that treatment of ICOS^{hi}CD4⁺ cells with vopratelimab further enhances IFNγ and TNFα expression, indicating activation of T cells.

### Example 5. ICOS Agonist Administration Increases Frequency of Tfh and B Cells and Enhances Antibody Responses

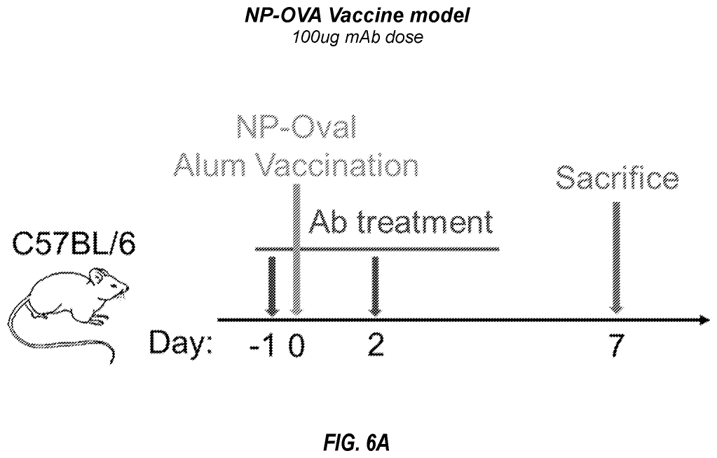

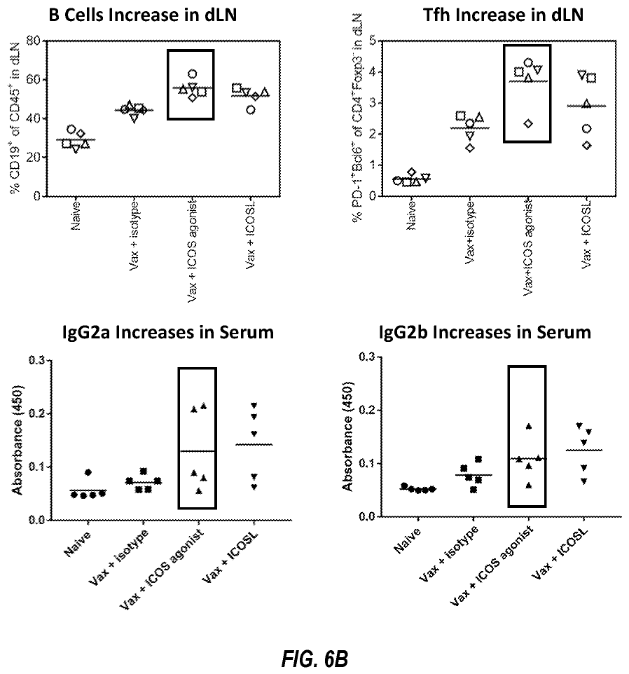

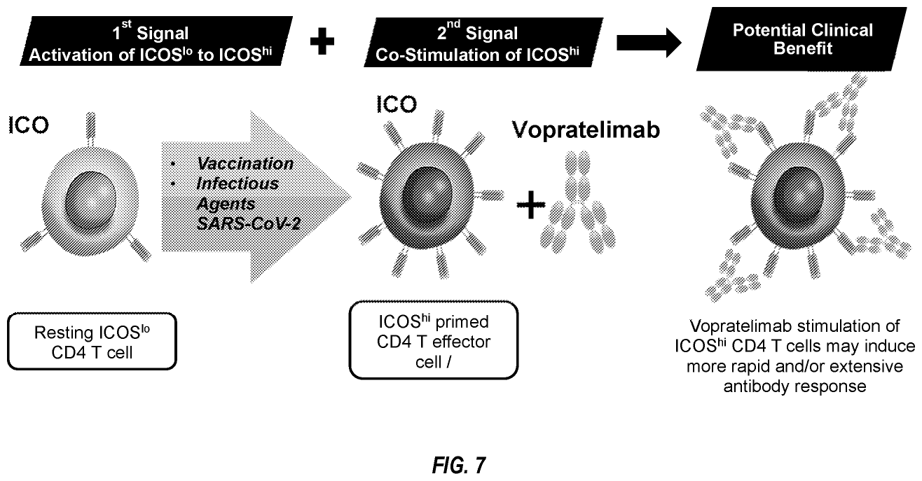

To assess the activity of ICOS agonist antibodies in inducing Tfh responses and antibody class switching, antibodies were tested in the NP-OVA vaccine model in C57BL/6 mice. As shown in FIG. 6A, mice were treated with NP-OVA and isotype matched antibody, ICOS agonist, or ICOS-L at 24 hours prior to and at 48 hours after vaccination. Mice were sacrificed 5 days following administration of the last treatment dose, and blood and dissected lymph node (dLN) were harvested. Tfh and B cell responses were assessed by flow cytometry, and OVA-specific antibodies were quantified by ELISA.

FIG. 6B shows treatment with ICOS agonist resulted in an increase in Tfh and B cell numbers within draining lymph nodes (top panels) as well as elevated serum IgG2a and IgG2b levels relative to both naïve and isotype treated animals (bottom panels). ICOSL was added as a positive control to assess agonist function.

### Example 6. ICOS Agonist Antibody Treatment Increases Antibody Responses Against Immunized SARS-CoV-2 Peptide Sequence

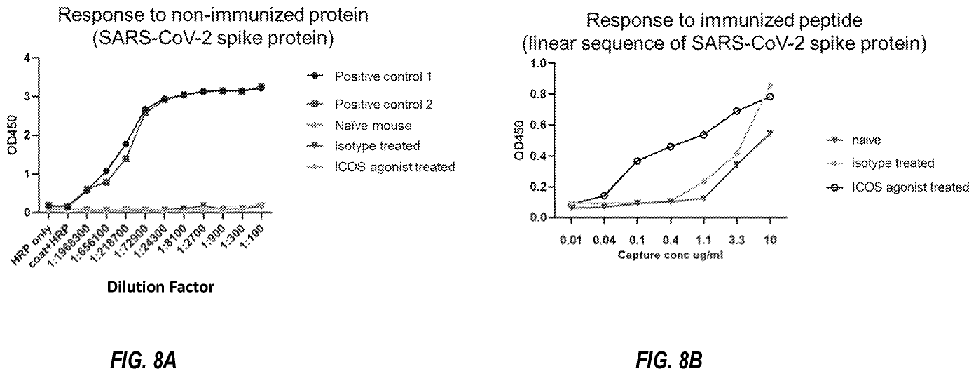

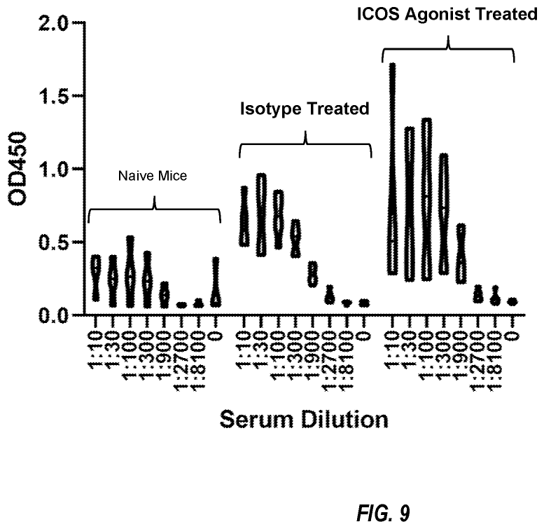

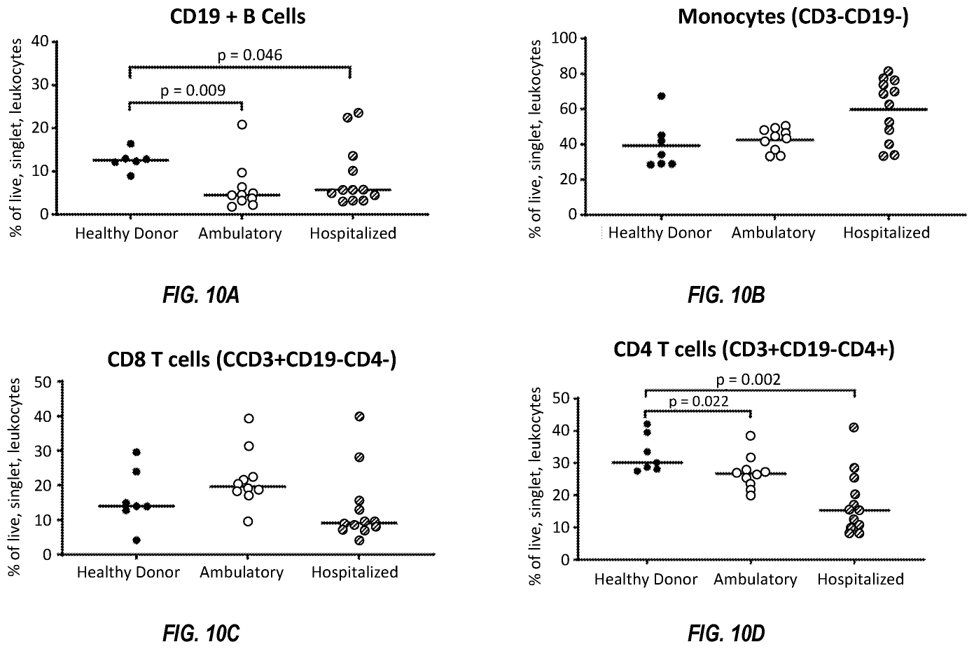

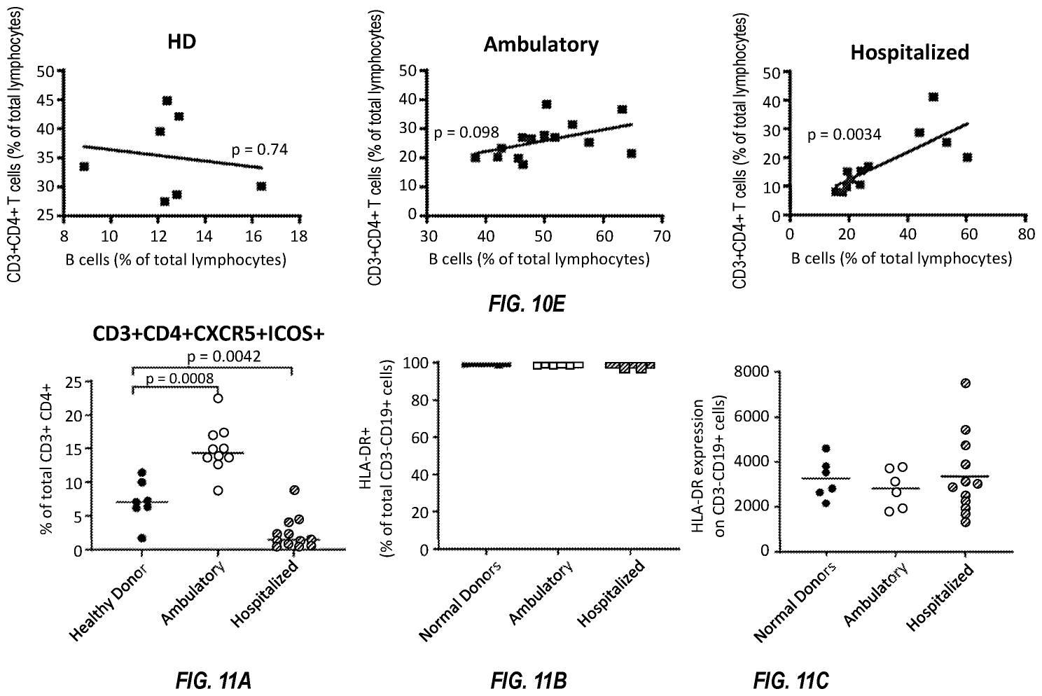

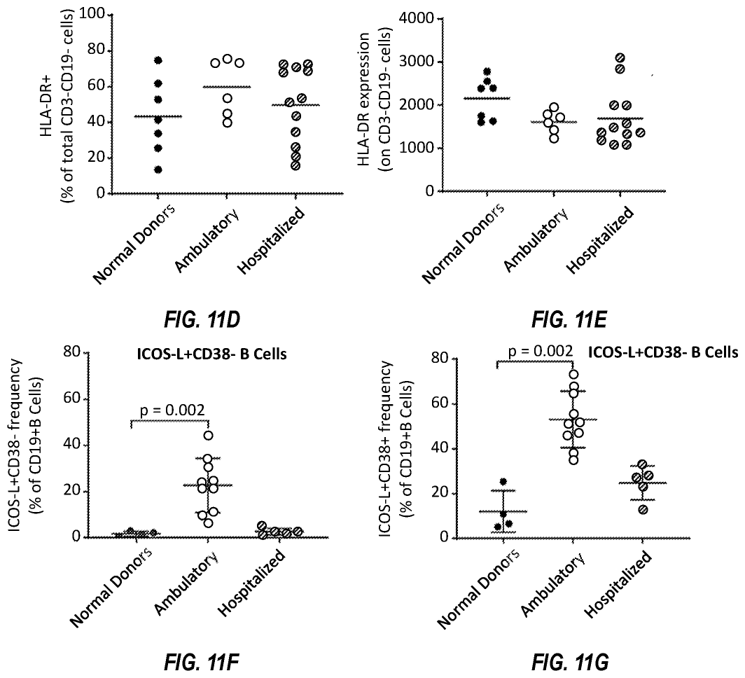

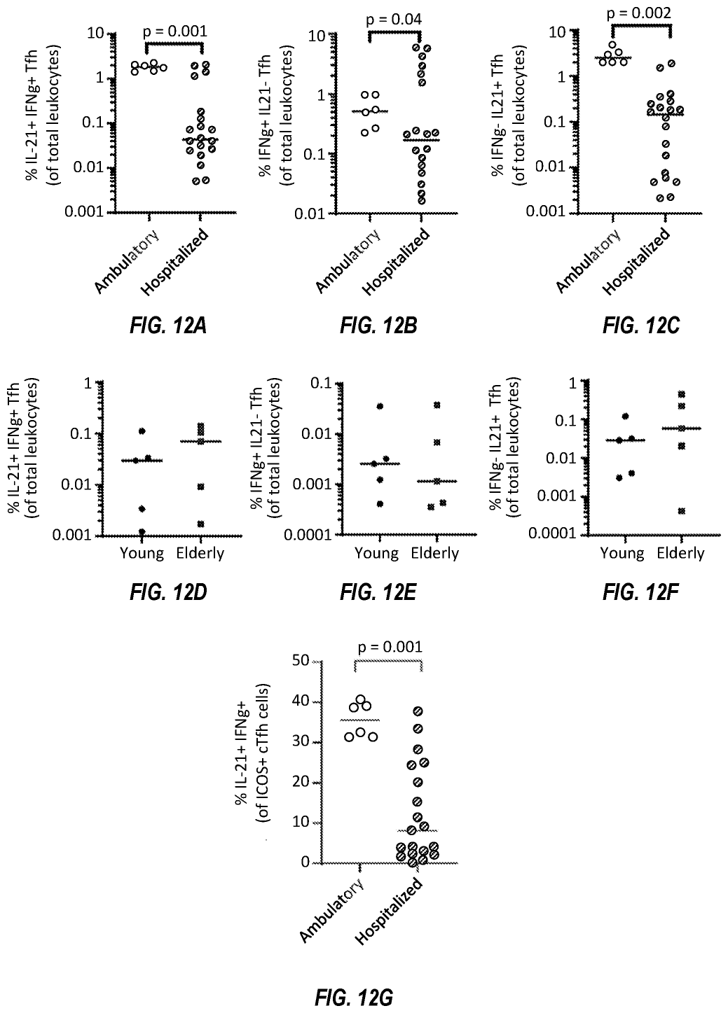

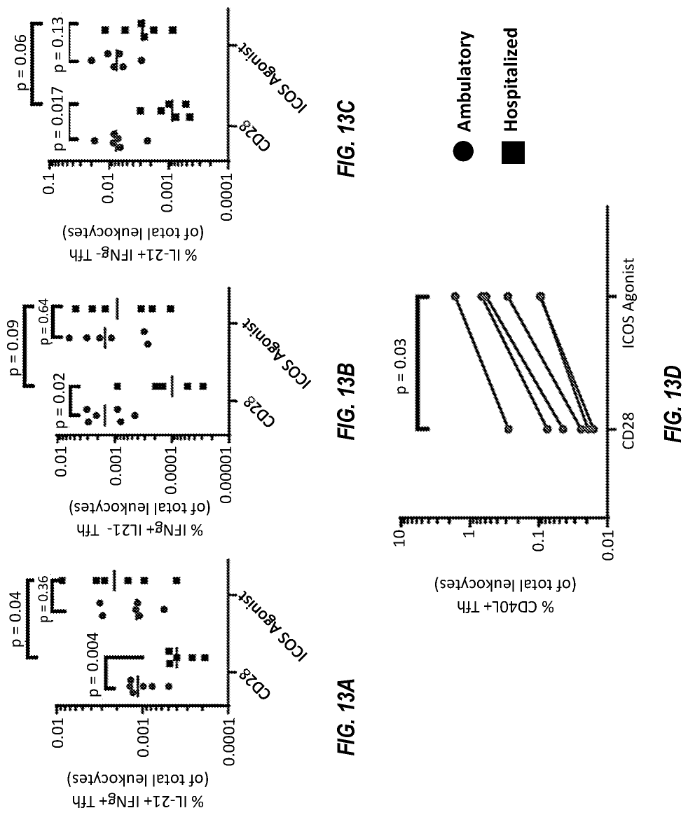

To assess the ability of an ICOS agonist antibody to increase antibody production in response to SARS-CoV-2, C57BL/6 mice were immunized with 50 μg of a linear peptide containing amino acids corresponding to the SARS-CoV-2 spike protein (Lifetein, LLC; Cat #LT5587) in complete Freund's adjuvant. Mice were treated with either isotype or ICOS agonist antibody (clone 36E10) 48 hours following immunization. Blood was collected 5 days after antibody treatment, and serum was assessed for antibodies against the immunized peptide (FIGS. 8B and 9), as well as a conformational epitope of SARS-CoV-2 (FIG. 8A), by ELISA. As shown, administration of ICOS agonist antibody increases antibody response against the immunized protein (linear SARS-CoV-2 spike protein) but not the conformational epitope, relative to immunized mice receiving an isotype control antibody or no antibody at all.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Sequences

| SEQ | | |
|---|---|---|
| ID | | |
| NO | Description | Sequence |
| 1 | Human ICOS precursor | MKSGLWYFFL FCLRIKVLTG EINGSANYEM FIFHNGGVQI |
| | (with signal sequence); | LCKYPDIVQQ FKMQLLKGGQ ILCDLTKTKG SGNTVSIKSL |
| | UniProtKB/Swiss-Prot: | KFCHSQLSNN SVSFFLYNLD HSHANYYFCN LSIFDPPPFK |
| | Q9Y6W8.1; 7 Jan. 2015 | VTLTGGYLHI YESQLCCQLK FWLPIGCAAF VVVCILGCIL |
| | | ICWLTKKKYS SSVHDPNGEY MFMRAVNTAK KSRLTDVTL |
| 2 | Human mature ICOS | EINGSANYEM FIFHNGGVQI LCKYPDIVQQ FKMQLLKGGQ |
| | (without signal sequence) | ILCDLTKTKG SGNTVSIKSL KFCHSQLSNN SVSFFLYNLD |
| | | HSHANYYFCN LSIFDPPPFK VTLTGGYLHI YESQLCCQLK |
| | | FWLPIGCAAF VVVCILGCIL ICWLTKKKYS SSVHDPNGEY |
| | | MFMRAVNTAK KSRLTDVTL |
| 3 | Mouse ICOS precursor | MKPYFCRVFV FCFLIRLLTG EINGSADHRM FSFHNGGVQI |
| | (with signal sequence); | SCKYPETVQQ LKMRLFRERE VLCELTKTKG SGNAVSIKNP |
| | UniProtKB/Swiss-Prot: | MLCLYHLSNN SVSFFLNNPD SSQGSYYFCS LSIFDPPPFQ |
| | Q9WVS0.2; 7 Jan. 2015 | ERNLSGGYLH IYESQLCCQL KLWLPVGCAA FVVVLLFGCI |
| | | LIIWFSKKKY GSSVHDPNSE YMFMAAVNTN KKSRLAGVTS |
| 4 | Mouse mature ICOS | EINGSADHRM FSFHNGGVQI SCKYPETVQQ LKMRLFRERE |
| | (without signal sequence) | VLCELTKTKG SGNAVSIKNP MLCLYHLSNN SVSFFLNNPD |
| | | SSQGSYYFCS LSIFDPPPFQ ERNLSGGYLH IYESQLCCQL |
| | | KLWLPVGCAA FVVVLLFGCI LIIWFSKKKY GSSVHDPNSE |
| | | YMFMAAVNTN KKSRLAGVTS |
| 5 | Cynomolgus monkey ICOS | MKSGLWYFFL FCLHMKVLTG EINGSANYEM FIFHNGGVQI |
| | precursor (with signal | LCKYPDIVQQ FKMQLLKGGQ ILCDLTKTKG SGNKVSIKSL |
| | sequence) | KFCHSQLSNN SVSFFLYNLD RSHANYYFCN LSIFDPPPFK |
| | | VTLTGGYLHI YESQLCCQLK FWLPIGCATF VVVCIFGCIL |
| | | ICWLTKKKYS STVHDPNGEY MFMRAVNTAK KSRLTGTTP |
| 6 | Cynomolgus mature ICOS | EINGSANYEM FIFHNGGVQI LCKYPDIVQQ FKMQLLKGGQ |
| | (without signal sequence) | ILCDLTKTKG SGNKVSIKSL KFCHSQLSNN SVSFFLYNLD |
| | | RSHANYYFCN LSIFDPPPFK VTLTGGYLHI YESQLCCQLK |
| | | FWLPIGCATF VVVCIFGCIL ICWLTKKKYS STVHDPNGEY |
| | | MFMRAVNTAK KSRLTGTTP |
| 7 | Vopratelimab heavy chain | EVQLVESGGG LVQPGGSLRL SCAASGFTFS DYWMDWVRQA |
| | variable region | PGKGLVWVSN IDEDGSITEY SPFVKGRFTI SRDNAKNTLY |
| | | LQMNSLRAED TAVYYCTRWG RFGFDSWGQG TLVTVSS |
| 8 | Vopratelimab light chain | DIVMTQSPDS LAVSLGERAT INCKSSQSLL SGSFNYLTWY |
| | variable region | QQKPGQPPKL LIFYASTRHT GVPDRFSGSG SGTDFTLTIS |
| | | SLQAEDVAVY YCHHHYNAPP TFGPGTKVDI K |
| 9 | Vopratelimab VH CDR1 | GFTFSDYWMD |
| 10 | Vopratelimab VH CDR2 | NIDEDGSITEYSPFVKG |
| 11 | Vopratelimab VH CDR3 | WGRFGFDS |
| 12 | Vopratelimab VL CDR1 | KSSQSLLSGSFNYLT |
| 13 | Vopratelimab VL CDR2 | YASTRHT |
| 14 | Vopratelimab VL CDR3 | HHHYNAPPT |
| 15 | Vopratelimab human IgG1 | EVQLVESGGG LVQPGGSLRL SCAASGFTFS DYWMDWVRQA |
| | heavy chain | PGKGLVWVSN IDEDGSITEY SPFVKGRFTI SRDNAKNTLY |
| | | LQMNSLRAED TAVYYCTRWG RFGFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPGK |
| 16 | Vopratelimab human κ | DIVMTQSPDS LAVSLGERAT INCKSSQSLL SGSFNYLTWY |
| | light chain | QQKPGQPPKL LIFYASTRHT GVPDRFSGSG SGTDFTLTIS |
| | | SLQAEDVAVY YCHHHYNAPP TFGPGTKVDI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| 17 | Vopratelimab human IgG1 | EVQLVESGGG LVQPGGSLRL SCAASGFTFS DYWMDWVRQA |
| | heavy chain V2 | PGKGLVWVSN IDEDGSITEY SPFVKGRFTI SRDNAKNTLY |
| | | LQMNSLRAED TAVYYCTRWG RFGFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPG |

What is claimed is:

1. A method of treating an infectious disease in a subject, said method comprising administering a therapeutically effective amount of an ICOS agonist to said subject.

2. A method of enhancing the effectiveness of a vaccine against an infectious disease in a subject, said method comprising administering a therapeutically effective amount of an ICOS agonist to said subject concurrently with or after administration of the vaccine to the subject.

3. The method of claim 2, wherein the ICOS agonist is administered after administration of the vaccine to the subject, such as after administration of a complete dose of vaccine.

4. The method of claim 2, wherein the ICOS agonist is administered concurrently with administration of the vaccine to the subject, such as concurrently with administration of each dose of vaccine.

5. The method of any one of claims 2-4, wherein the infectious disease is a bacterial disease.

6. The method of claim 5, wherein the bacterial disease is caused by infection with a bacteria selected from Staphylococcus aureus, Pseudomonas aeruginosa, Streptococcus pneumoniae, Corynebacterium diphtheriae (diphtheria), Clostridium tetani (tetanus), Borrelia bacterium (Lyme disease), Clostridium difficile, and Bordetella pertussis.

7. The method of claim 6, wherein the bacterial disease is caused by infection with Streptococcus pneumoniae.

8. The method of claim 7, wherein the vaccine is PneumoVax 34 (Merck).

9. The method of any one of claims 5-8, wherein the subject is at least 65 years old; or wherein the subject is younger than 65 years old and has a chronic illness, diabetes, alcoholism, weakened immune system, cochlear implants, CSF leaks, and/or is a smoker.

10. The method of any one of claims 5-9, wherein the subject has or has been diagnosed with an immune disorder selected from Wiskott-Aldrich syndrome, X-linked thrombocytopenia (XLT), CVID, hypogammaglobulinemia, idiopathic CD4 lymphopenia.

11. The method of any one of claims 1-4, wherein said infectious disease is a viral disease.

12. The method of claim 11, wherein said viral disease is caused by infection with a virus selected from an human papillomavirus (HPV), human immunodeficiency virus (HIV), dengue virus, zika virus, rotavirus, orthomyxovirus, coronavirus, adenovirus, herpesvirus, poxvirus, retrovirus, togavirus, hepadnavirus, varicella zoster virus, or influenza virus.

13. The method of claim 12, wherein said virus is a coronavirus selected from MERS-CoV, SARS-CoV, and SARS-CoV-2.

14. The method of claim 13, wherein said virus is SARS-CoV-2.

15. The method of claim 14, wherein said subject has tested positive for SARS-CoV-2.

16. The method of claim 14 or claim 15, wherein said subject has not exhibited symptoms of SARS-CoV-2 infection prior to said administering of the ICOS agonist.

17. The method of claim 14 or claim 15, wherein said subject has exhibited one or more symptoms of SARS-CoV-2, such as one or more of fever, cough, shortness of breath, loss of sense of taste and/or smell, fatigue, and body aches, prior to administering of the ICOS agonist.

18. The method of claim 17, wherein said symptoms include coughing, fever, and/or shortness of breath.

19. The method of any one of claims 11-18, wherein said subject has an immune disorder selected from Wiskott-Aldrich syndrome, X-linked thrombocytopenia (XLT), CVID, hypogammaglobulinemia, idiopathic CD4 lymphopenia.

20. The method of any one of claims 2-4, wherein said infectious disease is a viral disease caused by an influenza virus (e.g., influenza virus A, B, C, or D).

21. The method of claim 20, wherein the vaccine is a seasonal influenza vaccine or a pandemic influenza vaccine.

22. The method of claim 20 or claim 21, wherein the subject is at least 50 years old, at least 60 years old, or at least 65 years old.

23. The method of any one of claims 20-22, wherein said subject has an immune disorder selected from Wiskott-Aldrich syndrome, X-linked thrombocytopenia (XLT), CVID, hypogammaglobulinemia, idiopathic CD4 lymphopenia.

24. The method of any one of claims 2-4, wherein said infectious disease is a viral disease caused by varicella zoster virus.

25. The method of claim 24, wherein the vaccine is Zostavax (Merck).

26. The method of claim 24 or claim 25, wherein the subject is at least 50 years old, at least 60 years old, or at least 65 years old.

27. The method of any one of claims 24-26, wherein said subject has an immune disorder selected from Wiskott-Aldrich syndrome, X-linked thrombocytopenia (XLT), CVID, hypogammaglobulinemia, idiopathic CD4 lymphopenia.

28. The method of any one of claims 1-27, wherein said ICOS agonist is an anti-ICOS agonist antibody.

29. The method of claim 28, wherein said anti-ICOS agonist antibody is selected from vopratelimab, BMS-986226, KY1044, K Y1055, and GSK3359609, or an antibody comprising the heavy and light chain CDRs or the heavy and light chain variable regions of vopratelimab, BMS-986226, KY1044, KY1055, or CSK3359609.

30. The method of claim 28, wherein said anti-ICOS agonist antibody comprises:
a) a heavy and a light chain, wherein the heavy chain comprises a heavy chain CDR1 comprising the sequence of SEQ ID NO: 9, a heavy chain CDR2 comprising the sequence of SEQ ID NO: 10, and a heavy chain CDR3 comprising the sequence of SEQ ID NO: 11; and wherein the light chain comprises a light chain CDR1 comprising the sequence of SEQ ID NO: 12, a light chain CDR1 comprising the sequence of SEQ ID NO: 13, and a light chain CDR1 comprising the sequence of SEQ ID NO: 14;
b) a heavy and a light chain, wherein the heavy chain comprises a variable region comprising the amino acid sequence of SEQ ID NO: 7, and wherein the light chain comprises a variable region comprising the amino acid sequence of SEQ ID NO: 8; or
c) a heavy and a light chain, wherein the heavy chain comprises the amino acid sequence of SEQ ID NO: 15 or 17, and wherein the light chain comprises the amino acid sequence of SEQ ID NO: 16.

31. The method of claim 28, wherein said anti-ICOS agonist antibody is vopratelimab.

32. The method of any one of claims 28-31, wherein said anti-ICOS agonist antibody is administered at a dosage of from 0.01 to 0.3 mg/kg, from 0.03 to 0.1 mg/kg, or at a dosage of 0.3 mg/kg, 0.1 mg/kg, 0.03 mg/kg, or 0.01 mg/kg

33. The method of any one of claims 1-32, wherein said ICOS agonist is administered once every three weeks.

34. The method of claim 1-32, wherein said ICOS agonist is administered once.

35. The method of any one of claims 1-34, wherein said subject is at least 60 years old, at least 65 years old, at least 70 years old, at least 75 years old, or at least 80 years old.


SEQUENCE LISTING (17 sequences)

SEQ ID NO: 1 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 2 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 3 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 4 - protein, 180 aa, Mus musculus
EINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 5 - protein, 199 aa, Macaca fascicularis
MKSGLWYFFLFCLHMKVLTGEINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 6 - protein, 179 aa, Macaca fascicularis
EINGSANYEMFIFHNGGVQILCKYPDIVQQFKMQLLKGGQILCDLTKTKGSGNKVSIKSLKFCHSQLSNNSVSFFLYNLDRSHANYYFCNLSIFDPPPFKVTLTGGYLHIYESQLCCQLKFWLPIGCATFVVVCIFGCILICWLTKKKYSSTVHDPNGEYMFMRAVNTAKKSRLTGTTP

SEQ ID NO: 7 - protein, 117 aa, Artificial Sequence
  Vopratelimab heavy chain variable region
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 8 - protein, 111 aa, Artificial Sequence
  Vopratelimab light chain variable region
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 9 - protein, 10 aa, Artificial Sequence
  Vopratelimab VH CDR1
GFTFSDYWMD

SEQ ID NO: 10 - protein, 17 aa, Artificial Sequence
  Vopratelimab VH CDR2
NIDEDGSITEYSPFVKG

SEQ ID NO: 11 - protein, 8 aa, Artificial Sequence
  Vopratelimab VH CDR3
WGRFGFDS

SEQ ID NO: 12 - protein, 15 aa, Artificial Sequence
  Vopratelimab VL CDR1
KSSQSLLSGSFNYLT

SEQ ID NO: 13 - protein, 7 aa, Artificial Sequence
  Vopratelimab VL CDR2
YASTRHT

SEQ ID NO: 14 - protein, 9 aa, Artificial Sequence
  Vopratelimab VL CDR3
HHHYNAPPT

SEQ ID NO: 15 - protein, 447 aa, Artificial Sequence
  Vopratelimab human IgG1 heavy chain
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 218 aa, Artificial Sequence
  Vopratelimab human ? light chain
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 446 aa, Artificial Sequence
  Vopratelimab human IgG1 heavy chain V2
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG